(19) United States
(12) Patent Application Publication
(10) Pub. No.: US 2011/0009479 A1
(43) Pub. Date: Jan. 13, 2011

(54) Stimulating Neuronal Growth Using Brevetoxins

(75) Inventors: Daniel G. Baden, Wilmington, NC (US); Thomas F. Murray, Omaha, NE (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UNIVERSITY OF NORTH CAROLINA AT WILMINGTON, WILMINGTON, NC (US)

(21) Appl. No.: 12/597,125

(22) PCT Filed: Apr. 23, 2008
(86) PCT No.: PCT/US08/61254

§ 371 Date: Sep. 7, 2010

Related U.S. Application Data
(60) Provisional application No. 60/913,484, filed on Apr. 23, 2007.

(51) Int. Cl. A61K 31/35; A61K 31/366; A61P 25/28

(52) U.S. Cl. 514/450

(57) ABSTRACT

Disclosed are methods of treating neurodegenerative diseases or disorders in a subject in need of such treatment. The methods comprise administering to a subject in need of such treatment a therapeutically effective amount of brevetoxin or brevetoxin derivatives. Included in the diseases and disorders are Alzheimer's Disease, Huntington's Disease, Parkinson's Disease, Multiple sclerosis (MS), Amyotrophic Lateral Sclerosis (ALS/Lou Gehrig's Disease) and other Motor Neuron Diseases, Prion Diseases, Frontotemporal Dementia (FTD), and CNS dysfunctions such as schizophrenia, depression, and epilepsy. Also included are neurodegenerations resulting from stroke, heart attack, head and spinal cord trauma, traumatic brain injury, bleeding in the brain and other injuries to the central nervous system (CNS).

### CROSS-REFERENCE TO RELATED APPLICATIONS

This application claims priority of U.S. Provisional Application No. 60/913,484, filed Apr. 23, 2007.

### BACKGROUND OF THE INVENTION

1. Field of the Invention

The invention relates to methods of treating neurodegenerative diseases or disorders using brevetoxin derivative compounds or pharmaceutical formulations comprising the brevetoxin derivatives. The invention further relates to method of enhancing neuronal growth using brevetoxin derivatives or pharmaceutical formulations thereof.

2. Description of the Related Art

Neurodegenerative disorders and dysfunctions have different causes; however, they are all characterized by the dysfunction or loss of neurons and neuronal death. Such diseases and disorders are Alzheimer's Disease, Huntington's Disease, Parkinson's Disease, Multiple sclerosis (MS), Amyotrophic Lateral Sclerosis (ALS/Lou Gehrig's Disease) and other Motor Neuron Diseases, Prion Diseases, Frontotemporal Dementia (FTD); and CNS dysfunctions such as schizophrenia, depression, and epilepsy. Loss of neurons (neurodegeneration) is also associated with injuries to the central nervous system (CNS), stroke, heart attack, head and spinal cord trauma, traumatic brain injury, and bleeding in the brain.

Neurite initiation and growth is a critical process for the development of neurons and activity-dependent plasticity in neurons. Neurotransmitter reception initiates a number of biochemical signaling cascades in the neuron, one of which is the important elevation of intracellular calcium (Ghosh and Greenberg, Science 268:239-247, 1995; West, Proc. Natl. Acad. Sci. USA 98:11024-11031, 2001). Calcium influx induces dendritic and axonal growth through activation of the CaM-dependent protein kinase (CaMK) cascades, consisting of CaMK kinase (CaMKK) and its principal downstream targets CaMKI and CaMK IV (Soderling et al., Trends Biochem Sci 24:232-236, 1999; Means et al., Mol Endocrinol 14:4-13, 2000). Downstream effectors of CaMKI include the MAP-kinase pathway of Ras/MEK/ERK and the transcriptional factor cAMP-Response Element Binding protein (CREB) (Schmitt et al., J Neurosci 25:1281-1290, 2005), which derived expression of the pro-survival gene Bcl-2 (Bonni et al., Science 286:1358-1362, 1999; Riccio et al., Nat Neurosci 5:389-390, 1999). The Neurons has a CaMK, and CREB- and CBP-mediated gene expression to involve in the neurite growth (Gary et al., 2006; Redmond et al., Neuron 34:999-1010, 2002). The first step in calcium regulation of neurite growth is the influx of calcium entry into the cytoplasm. Entry calcium from different channels might lead to different Ca²⁺-dependent activation. In early development of neurons, the synaptically induced Ca²⁺ entry relied almost completely on N-methyl D-aspartate (NMDA) receptor activation (Yuste and Katz, Neuron 6:333-344, 1991). Through NMDAR, calcium plays a critical role in the induction of long term potentiation (LTP) (Perkel et al., Neuron 11:817-823, 1993) and in the transcription of many immediated-early genes (Cole et al., Nature 340:474-476, 1989). The other major route of calcium influx is through the Voltage-Gated Calcium Channels (L-type VGCCs). They transduce calcium-regulated signaling events to the nucleus tightly linked to gene expression (Catterall et al., Annu Rev Cell Dev Biol 16:521-555, 2000).

Voltage-gated sodium channels (VGSCs) are involved in the generation of action potentials in neurons. Numerous reports suggest that sodium is a critical intracellular second messenger that regulates in the nervous system (Yu, Nature 396:469-474, 2006). VGSCs represent the molecular target for several groups of neurotoxins. One such group of neurotoxins are brevetoxins. Brevetoxins were initially discovered when they were purified as toxins from cultures of the Florida red tide organism Karenia brevis also known as Gymnodinium breve and Ptychodiscus brevis (Baden, D. G., et al., Toxicon, 1982; 20(5):929-932). K. Brevis proliferates during red tide incidents. The brevetoxins, also known as “PbTx” toxins (Ptychodiscus brevis toxin), have since been characterized and found to be polycyclic-polyethers that initially were shown to have binding activity to a unique site associated with rat brain synaptosomes (Poli, M. A., et al., Molec. Pharm., 1986; 30:129-135). Brevetoxins are classified as neurotoxins that are known to bind to VGSCs. In particular, the effects of brevetoxins are mediated by interaction with receptor site 5 on the sodium channels. The general brevetoxin A and brevetoxin B backbone structure are as follows, with PbTx molecules (1-10) described.

### Brevetoxin B Backbone

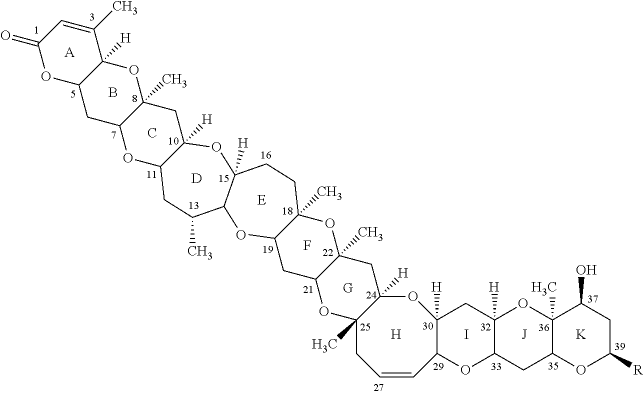

PbTx-2: R is CH₂C(═CH₂)CHO;

PbTx-3: R is CH₂C(═CH₂)CH₂OH;

PbTx-5: R is CH₂C(═CH₂)CHO, and OAc (instead of OH) at C37;

PbTx-6 R is CH₂C(═CH₂)CHO, and an epoxide at C27, C28 (instead of double bond);

PbTx-8 R is CH₂COCH₂Cl

PbTx-9 R is CH₂CH(CH₃)CH₂OH.

### Brevetoxin A Backbone

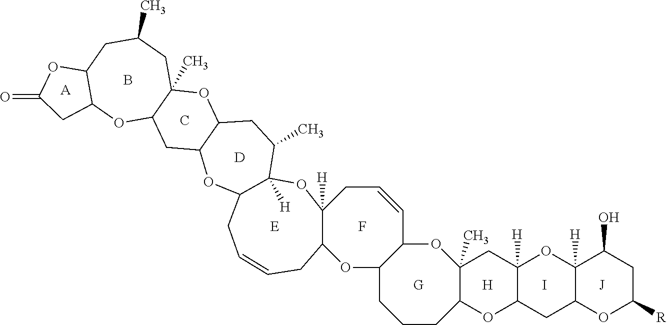

PbTx-1: R is CH₂C(═CH₂)CHO;

PbTx-7: R is CH₂C(═CH₂)CH₂OH;

PbTx-10 R is CH₂CH(CH₃)CH₂OH.

Brevetoxin effects VGSC channel and NMDA (LePage et al., Brain Res 959:120-127, 2003; Dravid et al., Brain Res 1031:30-38, 2005). Further, brevetoxin-induced neurite growth is triggered by VGSCs, following depolarization eliciting Ca²⁺-influx through NMDA channel which in turn activate Ca²⁺-dependent CaMKK signaling.

Brevetoxin and its derivatives can be employed to stimulate neuronal growth and to treat and/or prevent diagnose neuronal damage which occurs in certain neuronal disorders or neurodegenerative conditions.

Published U.S. Patent Application No. 20050124686 A1, the disclosure of which is incorporated herein by reference in its entirety, discloses the use of brevetoxin derivatives for treating diseases related to decreased mucus transport.

### SUMMARY OF THE INVENTION

The invention provides method of treatment of neurodegenerative diseases or disorders in a subject, comprising administering to a subject in need of such treatment a therapeutically effective amount of compounds or pharmaceutically acceptable salts, solvates, hydrates, complexes, or combinations thereof, of Formula (I):

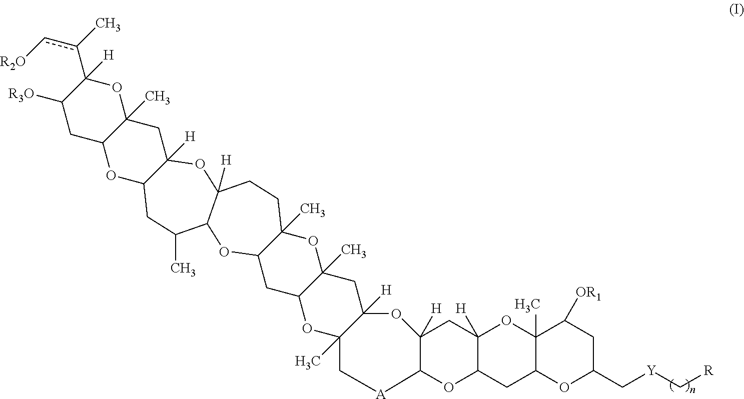

wherein
A is

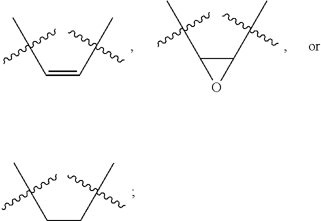

R is C₁-C₆ alkyl, C₂-C₆ alkenyl, C₃-C₈ cycloalkylcarbonyl, C₁-C₆ alkyl ester, C₂-C₆ alkenyl ester, —CHO, —CO₂H, amino, amido, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ halo alkyl, C₁-C₆ halo alkoxy, amino(C₁-C₆)alkyl, mono(C₁-C₆)alkylamino(C₁-C₆)alkyl or di(C₁-C₆)alkylamino(C₁-C₆)alkyl;
R₁ is H or —(CO)CH₃; and
R₂ and R₃ at each occurrence are independently —CH₂(CO)CH₃, —CH₂(CO)CH₂CH₃, —CH₂(CO)CH(CH₃)₂, —CH₂(CO)CH₂CH₂CH₃, —CH₂(CO)CH(CH₃)CH₂CH₃, or —CH₂(CO)CH₂CH(CH₃)₂,
or OR₂ and OR₃ can be taken together to form a six membered ring of the formula (Ia)

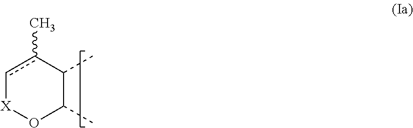

wherein X is C═O, CH₂, or CH(CH₃);
wherein the bracketed-dashed bonds indicate attachment to backbone;
Y is CH═CH, C═CH₂, C(O), CH(CH₃), or CH₂; and
   n is 0 or 1.

The invention also provides the method of treatment of neurodegenerative diseases or disorders using the compounds, or pharmaceutically acceptable salts, solvates, hydrates, complexes, or combinations thereof, of the Formula (II):

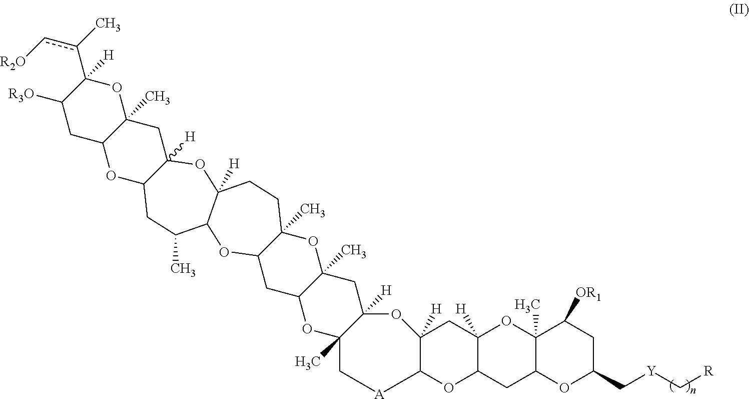

wherein
A is

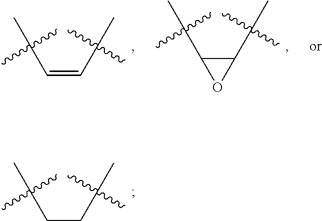

R is C₁-C₆ alkyl, C₂-C₆ alkenyl, C₁-C₆ alkyl ester, C₂-C₆ alkenyl ester, —CHO, —CO₂H, amino, amido, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
R₁ is H or —COCH₃; and
R₂ and R₃ at each occurrence are independently —CH₂COCH₃, —CH₂COCH₂CH₃, —CH₂COCH(CH₃)₂, —CH₂COCH₂CH₂CH₃, —CH₂COCH(CH₃)CH₂CH₃, or —CH₂COCH₂CH(CH₃)₂,
or OR₂ and OR₃ can be taken together to form a six membered ring of the formula (Ia)

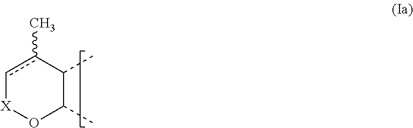

wherein X is C═O or CH(CH₃);
wherein the bracketed-dashed bonds indicate attachment to backbone;
Y is CH═CH, C═CH₂, C═O, or CH₂; and
n is 0 or 1.

Further, the invention provides method of treatment of neurodegenerative diseases or disorders using compounds, or pharmaceutically acceptable salts, solvates, hydrates, complexes, or combinations thereof, of the Formula (III):

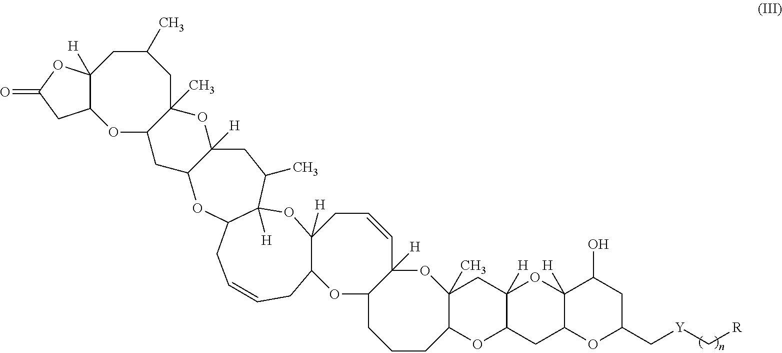

wherein
R is H, OH, halogen, C₁-C₆ lower alkyl, C₁-C₆ alkyl esters, C₂-C₆ alkenyl ester, amino, amido, —CHO, —CO₂H, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
n is 0 or 1; and
Y is C═O, CH═CH, C═CH₂, CH(CH₃) or CH₂.

Yet further, the instant invention provides method of treatment of neurodegenerative diseases or disorders using compounds, or pharmaceutically acceptable salts, solvates, hydrates, complexes, or combinations thereof, of the Formula (IV):

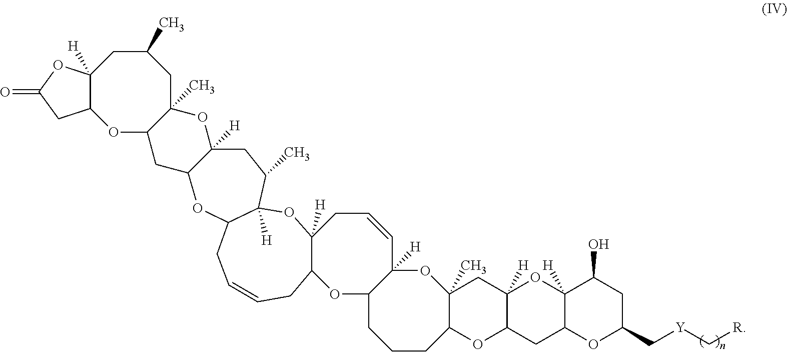

wherein
R is H, OH, halogen, C₁-C₆ lower alkyl, C₁-C₆ alkyl ester, C₂-C₆ alkenyl ester, amino, amido, —CHO, —CO₂H, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
n is 0 or 1; and
Y is CH═CH, C═CH₂, C═O, CH(CH₃), or CH₂.

The invention also provides method of treatment of neurodegenerative diseases or disorders using pharmaceutical formulations comprising a compound, or a pharmaceutically acceptable salt, solvate, hydrate, complex, or combinations thereof, of Formulas (I), (II), (III), and (IV) in combination with a pharmaceutically acceptable carrier, excipient, solvent, adjuvant or diluent.

The invention further provides method of treatment of Alzheimer's Disease, Huntington's Disease, Parkinson's Disease, Multiple sclerosis (MS), Amyotrophic Lateral Sclerosis (ALS/Lou Gehrig's Disease) and other Motor Neuron Diseases, Prion Diseases, Frontotemporal Dementia (FTD), and CNS dysfunctions such as schizophrenia, depression, and epilepsy in a subject, comprising administering to a subject in need of such treatment a therapeutically effective amount of compounds of Formulas (I), (II), (III), and (IV) or pharmaceutically acceptable salts, solvates, hydrates, complexes, or combinations thereof. The invention also provides a method of treatment of neurodegenerations resulting from stroke, heart attack, head and spinal cord trauma, traumatic brain injury, bleeding in the brain and other injuries to the central nervous system (CNS), using compound, or a pharmaceutically acceptable salt, solvate, hydrate, complex, or combinations thereof, of Formulas (I), (II), (III), and (IV).

Furthermore, the invention provides method of enhancing neuronal growth comprising administering to a subject, or contacting a cell with, a compound of Formulas (I), (II), (III), and (IV) in an amount effective to stimulate neuronal growth in the subject or cell, or a pharmaceutically acceptable salt, solvate, hydrate, complex, or combinations thereof.

### BRIEF DESCRIPTION OF THE DRAWINGS

FIG. 1 is showing brevetoxin concentration-response profile for neocortical neuron neurite growth. (A) Concentration-response quantification of 24 h neocortical neurons following 3 h exposure with either vehicle alone (control), or 1 nM, 3 nM, 10 nM, 30 nM, 100 nM, 300 nM, or 1000 nM of PbTx-2. Each value is mean±SEM of 120 cells. **p<0.01 and *p<0.05 compared to control group. (B) PbTx-2 concentration-response curve (generated using Prism 4). Biphasic concentration-response curve fit to data to quantify PbTx-2 EC₅₀ values. Images (C) and quantification (D) of neurite length in cortical neurons after 15 h, 24 h, 40 h and 108 h treatment by PbTx-2.

FIG. 2 is showing the role of voltage-gated sodium channels in PbTx-2-induced neurite growth. (A) representative images of 24 hr neocortical neurons following 3 hr treatment with either vehicle alone (control), 30 nM PbTx-2 alone, or in combination with 1 μM TTX (VGSC blocker) (Scale bar: 30 μm). (B) Neurite length quantified using PGP 9.5 antibody. Each value is mean±SEM of 30 cells. **p<0.01 compared to control group.

FIG. 3 is illustrating the influence of Ca²⁺ signaling pathways on PbTx-2-induced neurite growth. (A) Representative images of 24 hr neocortical neurons following 3 hr treatment with either vehicle alone (control), 30 nM PbTx-2 alone, or in combination with 1 μM MK801 (NMDAR blocker), STO-609 (CaMKK inhibitor), or nifedipine (voltage-sensitive calcium channels blocker) (Scale bar: 30 μm). (B) Neurite length quantified using PGP 9.5 antibody. Each value is mean±SEM of 30 cells. **p<0.01 compared to control group.

FIG. 4 is a schematic diagram summarizing the signaling molecular and pathway involve in neurite growth by PbTx-2-induced neocortical neurons.

### DETAILED DESCRIPTION OF THE INVENTION

### Definitions

Unless defined otherwise, all scientific and technical terms used herein have the same meaning as commonly understood by one of skill in the art to which this invention belongs.

All patents and publications referred to herein are hereby incorporated by reference for all purposes.

A “therapeutically effective” amount is defined as an amount effective to reduce or lessen at least one symptom of the disease being treated or to reduce or delay onset of one or more clinical markers or symptoms of the disease.

As used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

By “alkyl” and “C₁-C₆ alkyl” is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (e.g. of an alkyl, alkoxy or alkenyl group) is shorter or longer than 6 carbons, it will be so indicated in the second “C” as, for example, “C₁-C₁₀” indicates a maximum of 10 carbons. The alkyl groups herein are optionally substituted in one or more substitutable positions with various groups.

By the term “halogen” is meant fluorine, bromine, chlorine, and iodine.

“Alkenyl” and “C₂-C₆ alkenyl” means straight and branched hydrocarbon groups having from 2 to 6 carbon atoms and from one to three double bonds and includes, for example, ethenyl, propenyl, 1-but-3-enyl, 1-pent-3-enyl, 1-hex-5-enyl and the like. The alkenyl groups herein are optionally substituted in one or more substitutable positions with various groups.

As used herein, the term “cycloalkyl” refers to saturated carbocyclic groups having three to twelve carbon atoms. The cycloalkyl can be monocyclic, or a polycyclic fused system. Examples of such groups include cyclopropyl, cyclobutyl, cyclopentyl and cyclohexyl. The cycloalkyl groups herein are unsubstituted or, as specified, substituted in one or more substitutable positions with various groups. For example, such cycloalkyl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl), which is optionally mono-, di-, or trisubstituted. Preferred aryl groups of the invention are phenyl, 1-naphthyl, 2-naphthyl, indanyl, indenyl, dihydronaphthyl, tetralinyl or 6,7,8,9-tetrahydro-5H-benzo[α]cycloheptenyl. The aryl groups herein are unsubstituted or, as specified, substituted in one or more substitutable positions with various groups. Preferred aryl groups are optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

As used herein, the term “arylester” encompasses aryloxycarbonyl and arylcarbonyloxy groups. As used herein, the term “alkylester” encompasses alkyloxycarbonyl and alkylcarbonyloxy groups. As used herein, alkylcarbonyl carries the same meaning as alkanoyl. As used herein, the term “alkenylester” encompasses alkenyloxycarbonyl and alkenylcarbonyloxy groups.

As used herein, the term “alkylamide” encompasses alkylaminocarbonyl groups, dialkylcarbonyl groups, and alkanoylamino groups. As used herein, the term “alkenylamide” encompasses alkenylaminocarbonyl groups, dialkenylcarbonyl groups, and alkenylcarbonylamino groups.

The term alkylarylester as used herein refers to alkyloxycarbonyl and akanoyloxy groups in which the alkyl portion carries an aryl or heteroaryl group. The term alkenylarylester as used herein refers to alkenyloxycarbonyl and alkenylcarbonyloxy groups in which the alkenyl portion carries an aryl or heteroaryl group.

By “heteroaryl” is meant one or more aromatic ring systems of 5-, 6-, or 7-membered rings which includes fused ring systems of 9-11 atoms containing at least one and up to four heteroatoms selected from nitrogen, oxygen, or sulfur. Preferred heteroaryl groups of the invention include pyridinyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. The heteroaryl groups herein are unsubstituted or, as specified, substituted in one or more substitutable positions with various groups. Preferred heteroaryl groups are optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heterocycle”, “heterocycloalkyl” or “heterocyclyl” is meant one or more carbocyclic ring systems of 4-, 5-, 6-, or 7-membered rings which includes fused ring systems of 9-11 atoms containing at least one and up to four heteroatoms selected from nitrogen, oxygen, or sulfur. Preferred heterocycles of the invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide. The heterocycle groups herein are unsubstituted or, as specified, substituted in one or more substitutable positions with various groups. Preferred heterocycle groups are optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

In the methods of the invention, preferred compounds for use in the methods of are those where Y is C═CH₂, C═O, CHCH₃, or CH₂.

In one aspect the invention relates to the method of treatment of neurodegenerative diseases or disorders using compounds of Formula (I):

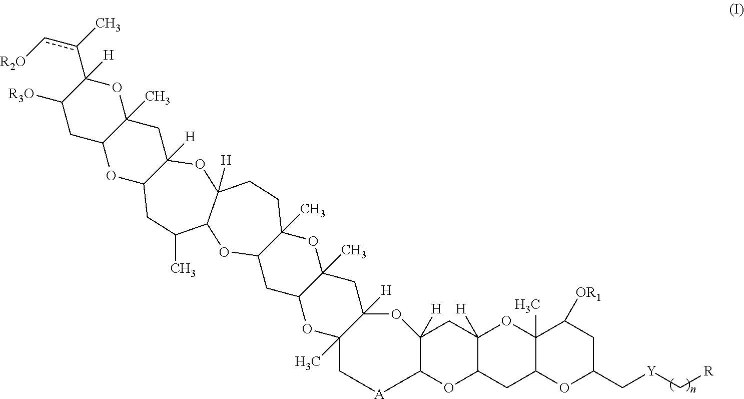

wherein
A is

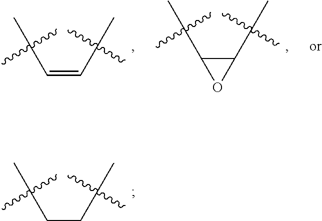

R is C₁-C₆ alkyl, C₂-C₆ alkenyl, C₁-C₆ alkyl esters, C₂-C₆ alkenyl esters, —CHO, —CO₂H, amines, amides, aryl esters, cycloalkyl esters, cycloalkenyl esters, purines, pyrimidines, heterocycle, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
R₁ is H or —(CO)CH₃; and
R₂ and R₃ at each occurrence are independently —CH₂(CO)CH₃, —CH₂(CO)CH₂CH₃, —CH₂(CO)CH(CH₃)₂, —CH₂(CO)CH₂CH₂CH₃, —CH₂(CO)CH(CH₃)CH₂CH₃, or —CH₂(CO)CH₂CH(CH₃)₂,
or OR₂ and OR₃ can be taken together to form a six membered ring of the formula (Ia)

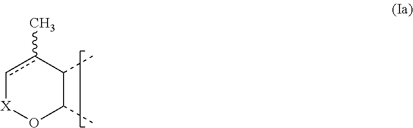

wherein X is C═O or CH(CH₃);
wherein the bracketed-dashed bonds indicate attachment to backbone; and
Y is CH═CH, C═CH₂, C═O, CHCH₃, or CH₂;
n is 0 or 1;
or a pharmaceutically acceptable salt, solvate, hydrate, complex, or combination thereof.

In a broad aspect, R is alkyl, alkyl ester, halogen, alkenyl, alkenyl ester, formyl, carboxyl, cycloalkyl, cycloalkyl ester, aryl, aryl ester, heteroaryl, heterocycle, heterocycloalkyl or heterocyclyl.

In another embodiment of this aspect, OR₂ and OR₃ are taken together to form a ring of formula (Ia), wherein the ring is

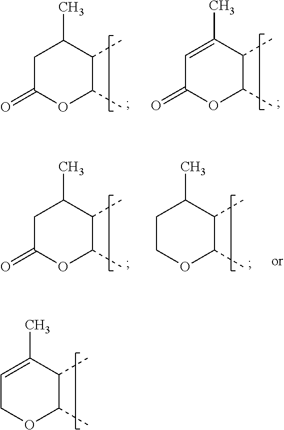

wherein the bracketed-dashed bonds indicate the point of attachment to the backbone.

In a preferred embodiment, the invention provides the method of treatment of neurodegenerative diseases or disorders using compounds of Formula (II):

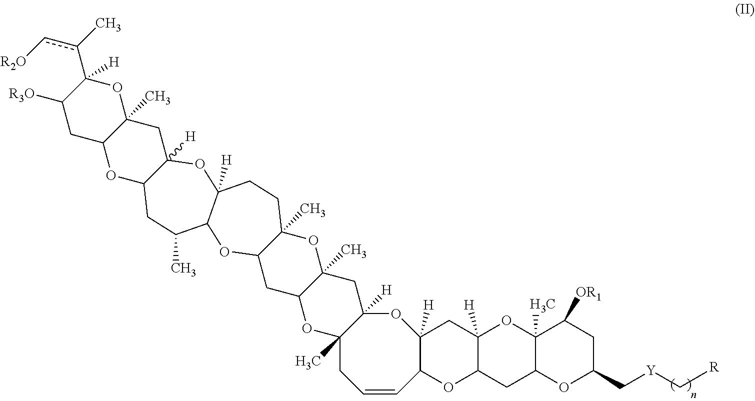

wherein
A is

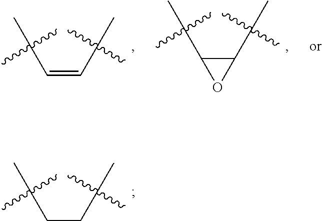

R is C₁-C₆ alkyl, C₂-C₆ alkenyl, C₁-C₆ alkyl esters, C₂-C₆ alkenyl esters, —CHO, —CO₂H, amines, amides, aryl esters, cycloalkyl esters, cycloalkenyl esters, purines, pyrimidines, heterocycle, or heteroaryl;
R₁ is H or —COCH₃; and
R₂ and R₃ at each occurrence are independently —CH₂COCH₃, —CH₂COCH₂CH₃, —CH₂COCH(CH₃)₂, —CH₂COCH₂CH₂CH₃, —CH₂COCH(CH₃)CH₂CH₃, or —CH₂COCH₂CH(CH₃)₂,
or OR₂ and OR₃ can be taken together to form a six membered ring of the formula (Ia)

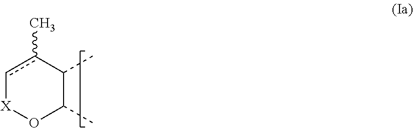

wherein X is C═O or CH(CH₃);
wherein the bracketed-dashed bonds indicate attachment to backbone;
Y is CH═CH, C═CH₂, C═O, or CH₂; and
n is 0 or 1
or a pharmaceutically acceptable salt, solvate, hydrate, complex, or combination thereof.

The invention also relates to the method of treatment of neurodegenerative diseases or disorders using compounds, or pharmaceutically acceptable salts, solvates, hydrates, complexes, or combination thereof, of Formula (III):

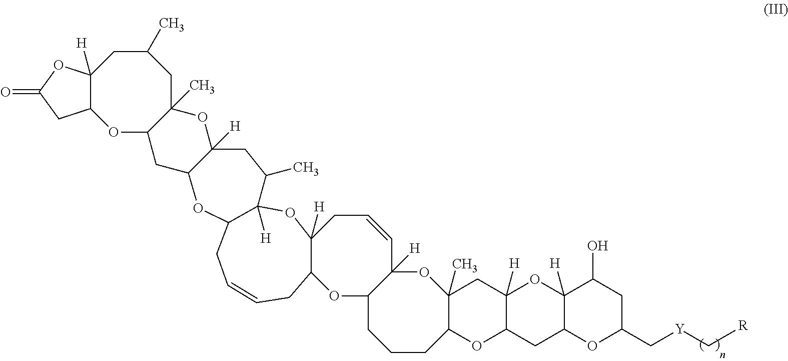

wherein
R is H, OH, halogen, C₁-C₆ lower alkyl, C₁-C₆ alkyl esters, C₂-C₆ alkenyl esters, amino, amido, formyl, carboxyl, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
Y is C═O, CH═CH, C═CH₂, CHCH₃ or CH₂; and
n is 0 or 1.

In one embodiment of this aspect, the compound of formula (III) is of formula (IV):

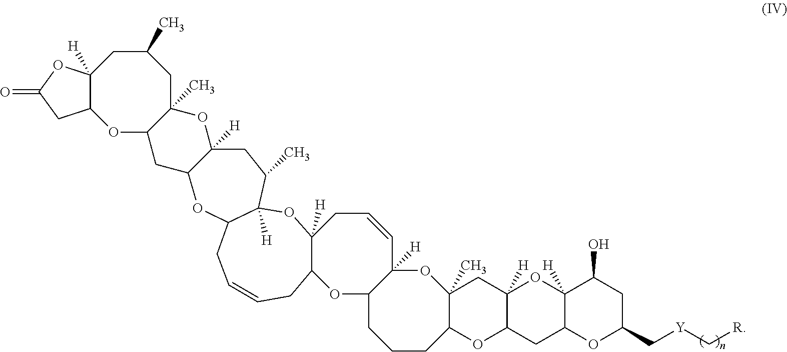

wherein
R is H, OH, halogen, C₁-C₆ lower alkyl, C₁-C₆ alkyl esters, C₂-C₆ alkenyl ester, amino, amido, formyl, carboxyl, aryl ester, cycloalkyl ester, cycloalkenyl ester, purinyl, pyrimidinyl, heterocyclyl, aryl, or heteroaryl;
Y is C═O, CH═CH, C═CH₂, CHCH₃ or CH₂; and
n is 0 or 1.

In even more preferred embodiment, the invention provides a method of treatment of neurodegenerative diseases or disorders using the following compounds:

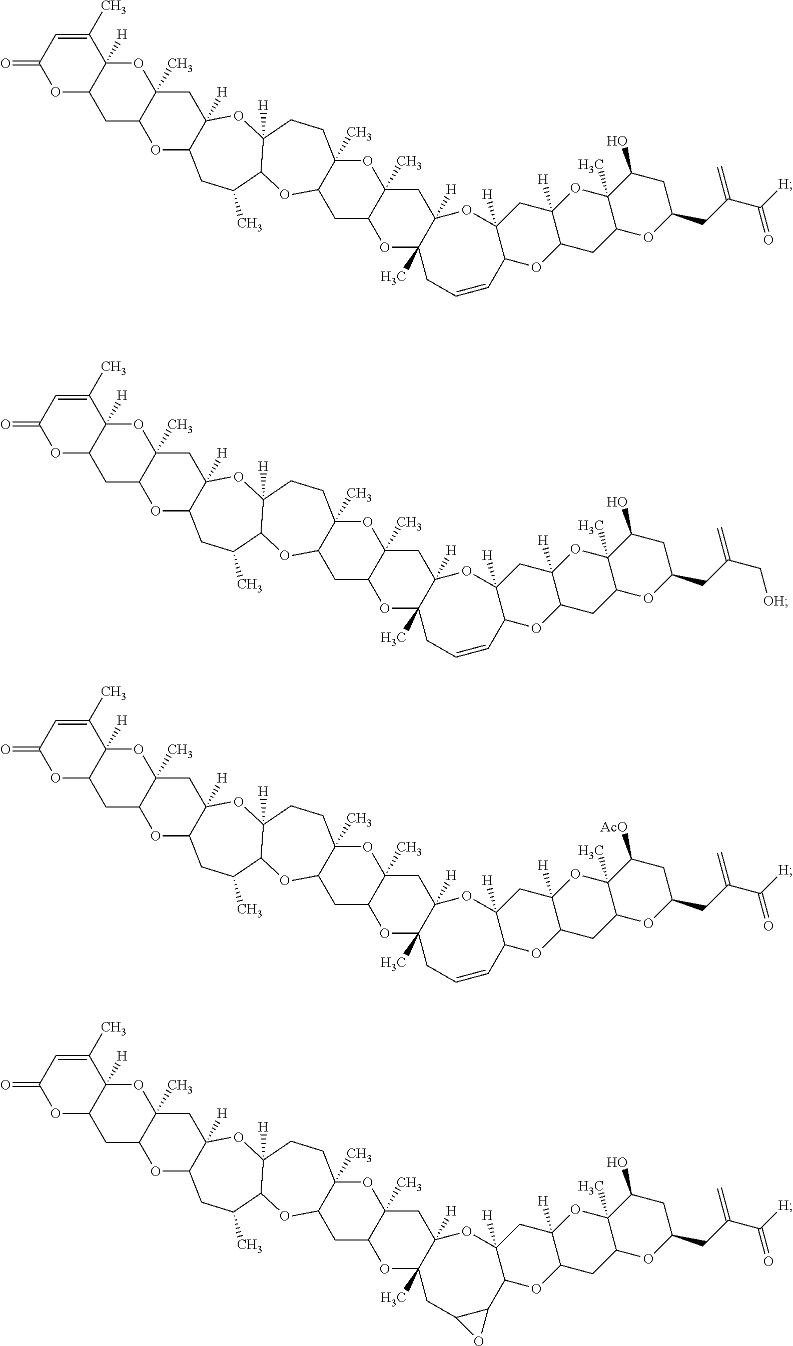

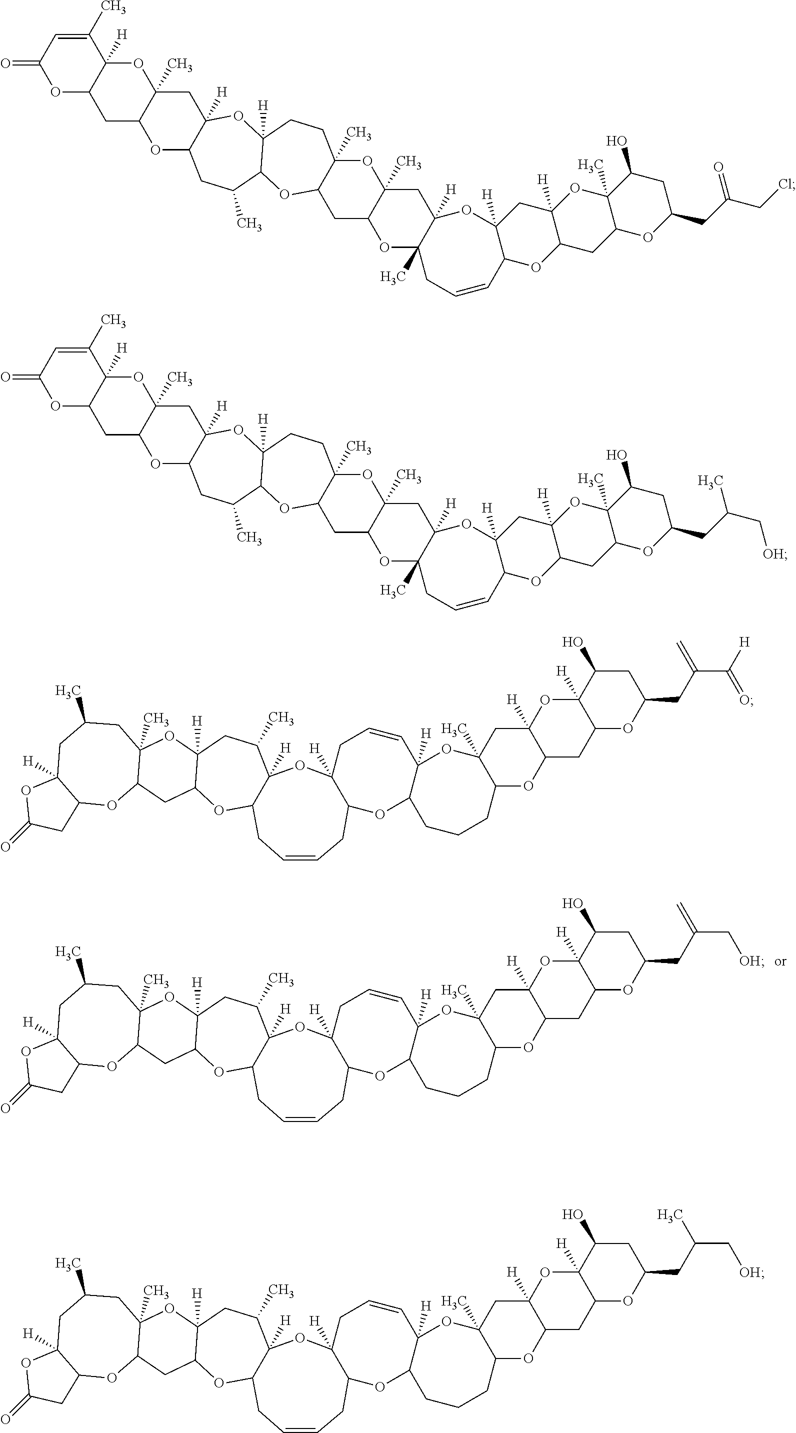

In another aspect, the invention provides methods of treatment of neurodegenerative diseases or disorders in a subject, comprising administering to a subject in need of such treatment a therapeutically effective amount of a compound having the formula, wherein the therapeutically effective concentration is between 1 and 300 nM.

In yet another aspect, the therapeutically effective concentration is between 10 and 100 nM.

In yet another aspect, the therapeutically effective concentration is between 20 and 40 nM.

In the preferred aspect, the therapeutically effective concentration is 30 nM.

In one embodiment, the invention provides method of treatment of Alzheimer's Disease, Huntington's Disease, Parkinson's Disease, Multiple sclerosis (MS), Amyotrophic Lateral Sclerosis (ALS/Lou Gehrig's Disease) and other Motor Neuron Diseases, Prion Diseases, Frontotemporal Dementia (FTD), and CNS dysfunctions such as schizophrenia, depression, and epilepsy, using compound, or a pharmaceutically acceptable salt, solvate, hydrate, complex, or combinations thereof, of Formulas (I), (II), (III), and (IV). The invention also provides a method of treatment of neurodegenerations resulting from stroke, heart attack, head and spinal cord trauma, traumatic brain injury, bleeding in the brain and other injuries to the central nervous system (CNS), using compound, or a pharmaceutically acceptable salt, solvate, hydrate, complex, or combinations thereof, of Formulas (I), (II), (III), and (IV).

In one embodiment, the method of treatment can be used to treat Alzheimer's Disease.

In another embodiment, the method of treatment can be used to treat Huntington's Disease.

In yet another embodiment, the method of treatment can be used to treat Parkinson's Disease.

In one embodiment, the method of treatment can be used to treat Multiple sclerosis (MS).

In one embodiment, the method of treatment can be used to treat Amyotrophic Lateral Sclerosis (ALS/Lou Gehrig's Disease) and other Motor Neuron Diseases.

In one embodiment, the method of treatment can be used to treat Prion Diseases.

In one embodiment, the method of treatment can be used to treat Frontotemporal Dementia (FTD).

In one embodiment, the method of treatment can be used to treat CNS dysfunctions such as schizophrenia, depression, and epilepsy.

In another aspect, the method of treatment can be used to treat neurodegenerations resulting from stroke, heart attack, head and spinal cord trauma, traumatic brain injury, and bleeding in the brain.

In yet another aspect, the method of treatment can be used to treat other injuries to the central nervous system (CNS).

In another aspect, the invention provides the method of treatment of Alzheimer's Disease, Huntington's Disease, Parkinson's Disease, Multiple sclerosis (MS), Amyotrophic Lateral Sclerosis (ALS/Lou Gehrig's Disease) and other Motor Neuron Diseases, Prion Diseases, Frontotemporal Dementia (FTD), and CNS dysfunctions such as schizophrenia, depression, and epilepsy, using compound, or a pharmaceutically acceptable salt, solvate, hydrate, complex, or combinations thereof, of Formulas (I), (II), (III), and (IV). The invention also provides a method of treatment of neurodegenerations resulting from stroke, heart attack, head and spinal cord trauma, traumatic brain injury, bleeding in the brain and other injuries to the central nervous system (CNS) in a subject, comprising administering to a subject in need of such treatment a therapeutically effective amount of a compound having the formula, wherein the therapeutically effective concentration is between 1 and 300 nM.

In yet another aspect, the therapeutically effective concentration is between 10 and 100 nM.

In yet another aspect, the therapeutically effective concentration is between 20 and 40 nM.

In the preferred aspect, the therapeutically effective concentration is 30 nM.

In another aspect, the invention provides methods for enhancing neuronal growth comprising administering to a subject, or contacting a cell with, a compound of the invention, or a pharmaceutically acceptable salt, solvate, hydrate, complex, or combination thereof, in an amount effective to stimulate neuronal growth in the subject or cell.

In more preferred aspect, the invention provides method for enhancing neuronal growth comprising administering to a subject, or contacting a cell with, the following compounds:

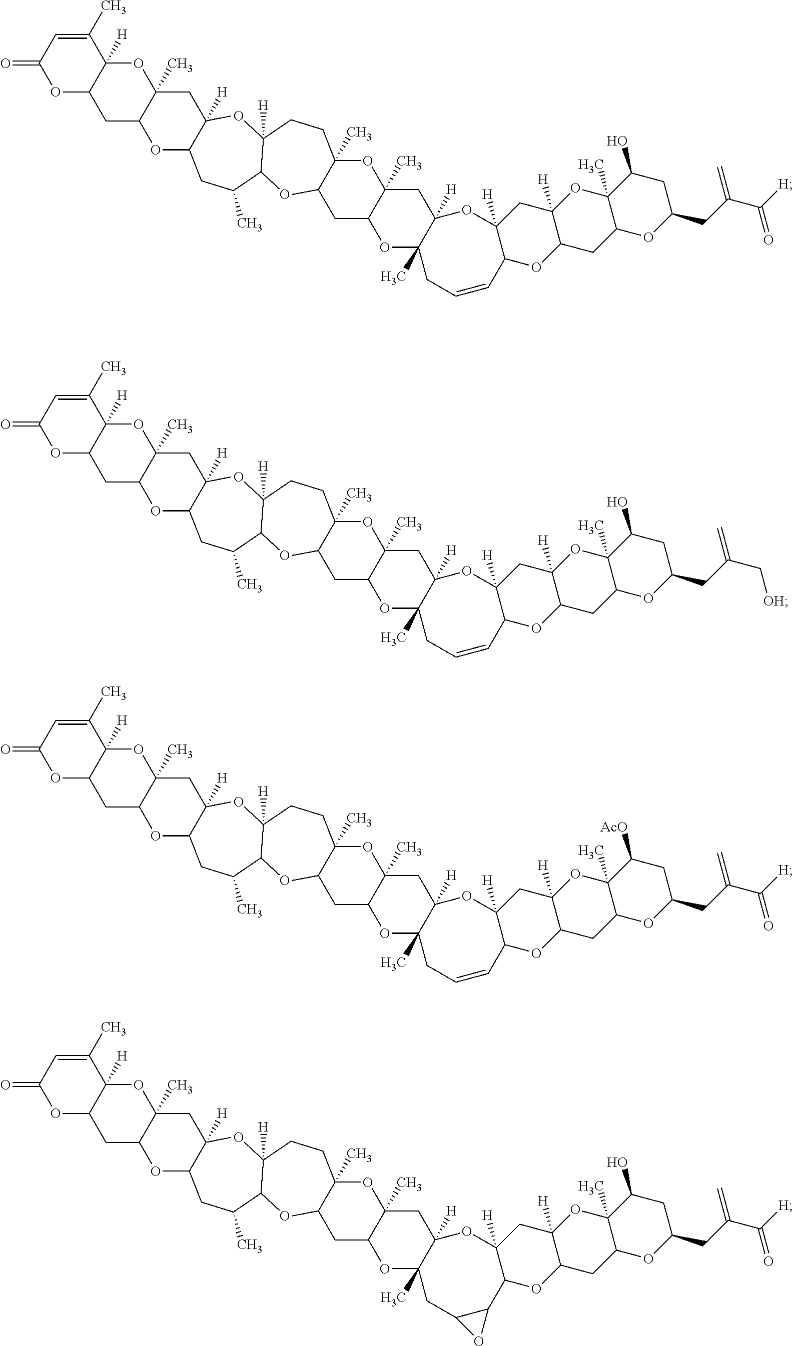

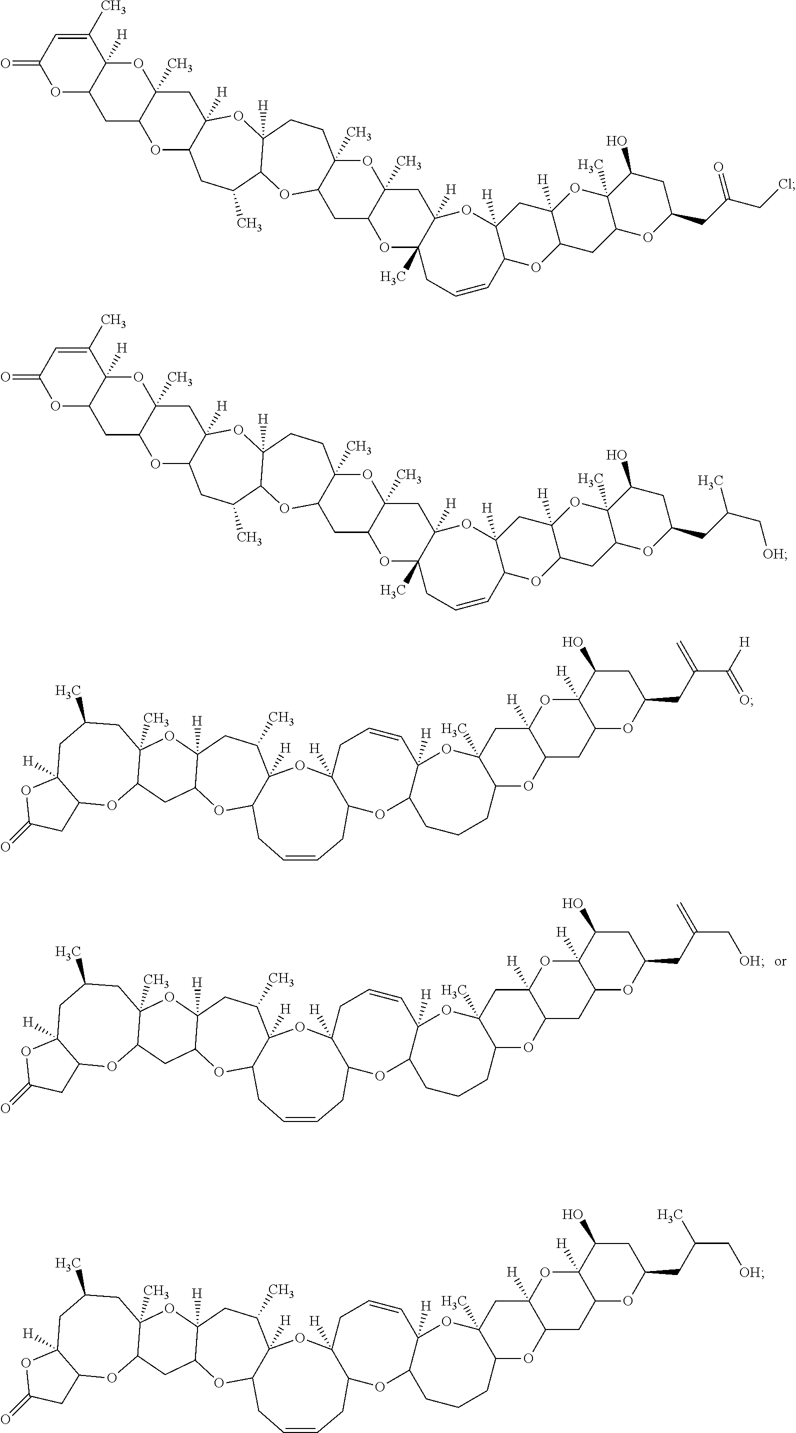

In another aspect, the invention provides the method for enhancing neuronal growth comprising administering to a subject, or contacting a cell with, a compound of the invention, or a pharmaceutically acceptable salt, solvate, hydrate, complex, or combination thereof, in an amount effective to stimulate neuronal growth in the subject or cell, wherein the effective amount has the concentration between 1 and 300 nM.

In yet another aspect, the therapeutically effective concentration is between 10 and 100 nM.

In yet another aspect, the therapeutically effective concentration is between 20 and 40 nM.

In the preferred aspect, the therapeutically effective concentration is 30 nM.

In another embodiment of this aspect, the method can optionally comprise in combination with the compound of Formula (I)-(IV) or a pharmaceutically acceptable salt, solvate, hydrate, complex, or combination thereof, an effective amount of a compound known to be useful for the treatment of conditions or diseases associated with dysfunction and loss of specific groups of neurons. The methods of the invention can optionally comprise additional therapeutic regimen such as supportive or adjuvant therapy.

In one embodiment of the methods of the invention, the subject is a mammal. In a more preferred embodiment, the mammal is a human.

The methods of invention employ therapeutically effective amounts. In a proffered aspect, the therapeutically effective concentration is non-lethal.

In another aspect, the therapeutically effective concentration is nontoxic.

The methods of the invention employ therapeutically effective amounts: for inhalation, oral, parenteral, sublingual, intranasal, intrathecal, depo, implants, topical, and rectal administration from about 0.1 pg/day to about 100 mg/day. The therapeutically effective amounts will vary according to various parameters including, for example, the route of administration, the distribution of the compound, the metabolism of the compound, the excretion of the compound, the particular therapeutic use, and the physical characteristics of the subject/patient, and are well within the knowledge of those skilled in the art.

In a preferred aspect, the therapeutically effective amounts for oral non-inhalation administration is about 1 mg/day to about 100 mg/day

In a preferred aspect, the therapeutically effective amounts for parenteral, and depo administration is from about 1 pg/day to about 100 mg/day.

The invention also includes the use of a compound of Formula (I)-(IV), or a pharmaceutically acceptable salt, solvate, hydrate, complex, or combination thereof for the manufacture of a medicament for use in treating a subject who has a neurodegenerative disease, or in preventing a subject from developing, neurodegenerative diseases or disorders, and symptoms associated with those disorders or diseases, and who is in need of such treatment.

In one aspect, this use of a compound of formula (I)-(IV) can be employed where the disease or condition is Alzheimer's Disease.

In another aspect, this use of a compound of formula (I)-(IV) can be employed where the disease or condition is Huntington's Disease.

In yet another aspect, this use of a compound of formula (I)-(IV) can be employed where the disease or condition is Parkinson's Disease.

In another aspect, this use of a compound of formula (I)-(IV) can be employed where the disease or condition is Multiple sclerosis (MS).

In another aspect, this use of a compound of formula (I)-(IV) can be employed where the disease or condition is Amyotrophic Lateral Sclerosis (ALS) or other Motor Neuron Disease.

In another aspect, this use of a compound of formula (I)-(IV) can be employed where the disease or condition is Prion Disease.

In yet another aspect, this use of a compound of formula (I)-(IV) can be employed where the disease or condition is Frontotemporal Dementia (FTD).

In one aspect, this use of a compound of formula (I)-(IV) can be employed where the disease or condition is CNS dysfunction such as schizophrenia, depression, and epilepsy.

In another aspect, this use of a compound of formula (I)-(IV) can be employed where the disease or condition resulting from stroke, heart attack, head and spinal cord trauma, traumatic brain injury, or bleeding in the brain.

In yet another aspect, this use of a compound of formula (I)-(IV) can be employed where the disease or condition resulting from other injuries to the central nervous system (CNS).

The compounds of the invention, pharmaceutical formulations comprising said compounds, and pharmaceutically acceptable salts thereof, are useful for treating a subject, preferably a mammal, more preferably a human, suffering from a neurodegenerative disease associated with dysfunction and loss of specific groups of neurons, and are useful for helping to prevent or delay the onset of such a disease or condition. The compounds and formulations of the invention are particularly useful for treating, preventing, or slowing the progression of Alzheimer's Disease, Huntington's Disease, Parkinson's Disease, Multiple sclerosis (MS), Amyotrophic Lateral Sclerosis (ALS/Lou Gehrig's Disease) and other Motor Neuron Diseases, Prion Diseases, Frontotemporal Dementia (FTD), CNS dysfunctions such as schizophrenia, depression, and epilepsy, and neurodegenerations resulting from stroke, heart attack, head and spinal cord trauma, traumatic brain injury, bleeding in the brain and other injuries to the central nervous system (CNS). When treating or preventing a disease and condition associated with dysfunction and loss of specific groups of neurons, and the associated symptoms, the compounds of the invention can either be used individually or in combination, as is best for the subject.

With regard to these diseases and conditions, the term “treating” means that compounds of the invention can be used in subjects, preferably human subjects/patients, with existing condition or disease. The compounds of the invention will not necessarily cure the subject who has the disease but will delay or slow the progression or prevent further progression of the disease thereby giving the individual a more useful life span.

The term “preventing” means that that if the compounds of the invention are administered to those who do not now have the disease, or symptom(s) of the condition, but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease. By delaying the onset of the disease, compounds of the invention can prevent the individual from getting the disease during the period in which the individual would normally have gotten the disease or reduce the rate of development of the disease or some of its effects but for the administration of compounds of the invention up to the time the individual ultimately gets the disease. Preventing also includes administration of the compounds of the invention to those individuals thought to have predisposition for the disease.

In a preferred aspect, the compounds of the invention are useful for slowing the progression of disease symptoms.

In another preferred aspect, the compounds of the invention are useful for preventing the further progression of disease symptoms.

In treating or preventing the above diseases, the compounds of the invention are administered in a therapeutically effective amount. The therapeutically effective amount will vary depending on the particular compound used, the physical characteristics of the subject to be treated, and the route of administration, as is known to those skilled in the art.

In treating a subject displaying any of the diagnosed above conditions a physician may administer a compound of the invention immediately and continue administration indefinitely, as needed.

Various in vivo and in vitro models can be employed to evaluate the compounds of formulas (I)-(IV) in the method of the invention Such models include in vitro assays in mammalian neuron culture to assess the influence of the compounds on neuronal growth, neurite extension, axonal extension, dendritic branching and growth cone motility. In vivo models include evaluation of the compounds of the invention in knockout mice and knockout rats.

The compounds of the invention can be administered orally, parenterally, (IV, IM, depo-IM, SQ, and depo SQ), sublingually, intranasally, by inhalation, intrathecally, topically, vaginally, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as aerosols, inhalants, tablets, capsules, or elixirs for oral administration or in sterile solutions or suspensions for parenteral administration. Typically the compounds described above are formulated into pharmaceutical compositions using techniques and procedures well known in the art.

About 0.1 pg to about 100 mg of a compound or mixture of compounds of the invention or a physiologically acceptable salt, solvate, hydrate, complex, ester, or combination thereof, is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 0.1 pg to about 100 mg, preferably about 1 pg to about 1 μg for inhalation administration, preferably about 100 ng to about 1 mg for injection/intravenous administration, or about 1 mg to about 100 mg for oral administration (e.g., tablets, elixirs, capsules, etc.), of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare pharmaceutical compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed or blended with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts, solvates, hydrates, complexes, or prodrugs may also be used in formulating effective pharmaceutical compositions.

The concentration of the compound is effective for delivery of an amount upon administration that lessens or ameliorates at least one symptom of the poisoning or disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, route of administration, metabolism, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral, non-inhalation administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed or blended with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered by inhalation, orally or intranasally, parenterally (IV, IM, depo-IM, SQ, and depo-SQ), sublingually, intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the subject 1, 2, 3, or 4, or as needed, times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

Depending on whether asymmetric carbon atoms are present, the compounds of the invention can be present as mixtures of isomers, as racemates, or in the form of pure isomers.

Salts of compounds are preferably the pharmaceutically acceptable or non-toxic salts of compounds of Formula (I)-(IV). For isolation and purification purposes it is also possible to use pharmaceutically unacceptable salts.

### Synthesis of Compounds

Various synthetic methodologies can be used to make compounds of the invention; certain of the brevetoxins are suitable starting materials. Suitable methodologies are known in the art. Representative synthetic procedures for preparing compounds of the invention from such starting materials are disclosed in, e.g., Mende, T. J., et al., Tetr. Lett., 1990; 31(37):5307-5310; Trainer, V. L., et al., Molec. Pharm., 1991; 40(6):988-994; Keck, G. E., et al., Tetrahedron Lett., 1987, 28:139-142; Alvarez, E., et al., Chem. Rev., 1995, 95:1953-1980; Rein, et al., 1994: (a) J. Org. Chem., 59:2107-2113; (b) J. Org. Chem., 59:2101-2106. Each of these references is incorporated herein by reference in its entirety. Those skilled in the art will appreciate that minor modifications can be made to the particular procedures to arrive at compounds of the invention.

The disclosures in this application of all articles and references, including patents, are incorporated herein by reference in their entirety.

The invention is illustrated further by the following examples which are not to be construed as limiting the invention in scope or spirit to the specific procedures and compounds described in them.

### EXAMPLES

General. Primary cell cultures of neocortical neurons were obtained from Swiss-Webster mice on embryonic day 16. Embryos were extracted following euthanasia via CO₂ asphyxiation and their neocortices were collected. Isolated neocortices were then removed of their meninges, minced by trituration using a Pasteur pipette, and treated with trypsin for 20 minutes at 37° C. The cells were further dissociated via two successive trituration and sedimentation steps in isolation buffer containing soybean trypsin inhibitor and DNase. The cells underwent another centrifugation step and were resuspended in a neuron-plating medium containing Eagles's minimal essential medium with Earle's salt (MEM) (Cell Signaling Technology, Beverly, Mass.), along with 2 mM L-glutamine, 10% fetal bovine serum, 10% horse serum, 100 I.U./ml penicillin and 0.10 mg/ml streptomycin, pH 7.4. Cells were plated onto poly-L-lysine treated coverslips, 24-wells culture plates at a density of 0.1×10⁶ cells/well. Plates were incubated at 37° C. with 5% CO₂ and 95% humidity. On day 2, post-plating, cells were treated with Cytosine arabinoside (10 μM) to prevent proliferation of non-neuronal cells. All animal use protocols were approved by the Institutional Animal Care and Use Committee (IACUC).

### Example 1

Effects of brevetoxin on neocortical neurite growth. Culture cells were exposed to brevetoxin (Tocris Cookson, Inc, Ellisville, Mo.) (1 nM, 3 nM, 10 nM, 30 nM, 100 nM, 300 nM, 1000 nM and untreated) and in combination with VGSC antagonist TTX (1 μM) (Tocris Cookson, Inc, Ellisville, Mo.), NMDA receptor antagonist MK801 (1 μM) (Sigma), VGCC antagonist nifedipine (1 μM) (Sigma) and CaMKK inhibitor STO-609 (2.6 μM) (Calbiochem, San Diego, Calif.). For each experimental protocol, cells were kept in the above-mentioned culture medium for 3 h following plating. Brevetoxin and others antognists were then administered into the culture medium for a defined period as indicated in the result and corresponding figures.

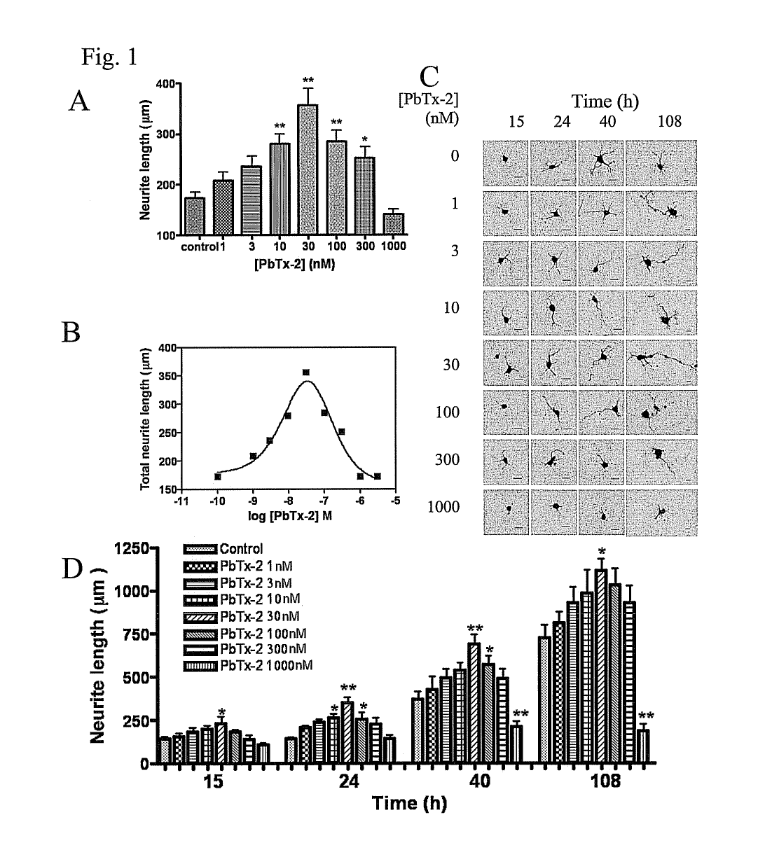

PbTx-2 concentration-response for neocortical neurite growth was quantified. 24 hr after treatment. Total neurite length was 279.9±19.98 μm for 10 nM of PbTx-2, 357.0±32.15 μm for 30 nM, 284.6±22.6 μm for 100 nM, and 251.9±22.38 μm for 300 nM of PbTx-2, which was significantly longer than control (173.1±13.16 μm) (FIG. 1 A). To obtain the binding curve, which is illustrated in FIG. 1 B, the following equation was used:

Y=Bottom+(Top-Bottom)/(1+10̂((LogEC₅₀1−X)*HillSlope))+(Top-Bottom2)/(1+10̂((LogEC₅₀2−X)*HillSlope2))

wherein Top and Bottom are fixed the same, LogEC₅₀1 and LogEC₅₀2 are the midpoint potency parameters for the two different phases, respectively, HillSlope1 and HillSlope2 are their corresponding Hill slopes

Further analysis of concentration-response and time-response data showed that 30 nM PbTx-2 has significant effect on stimulate total neurite growth in the time profile of 15 h, 24 h, 40 h, 108 h than the control (about 1.5 fold for 15 h, p<0.05; 2.0 fold for 24 h, p<0.01; 1.8 fold for 40 h, p<0.01; 1.4 fold for 108 h, p<0.05, FIG. 1 D).

### Example 2

Immunocytochemistry. After treatment, cells were allowed to grow for varying times (15 hr, 24 hr, 40 hr and 108 hr), media was aspirated, and cells were fixed with ice-cold 4% paraformaldehyde in 0.1 M phosphate buffer (pH 7.4) for 0.5 hour at 4° C. Then, they were rinsed in ice-cold 0.01% Triton X-100 buffer for 5 min at 4° C. two times, washed in PBS for 5 min three times, and incubated 30 min at 4° C. with PBS containing 2% horse serum. Aspirate and carefully transfer coverslips to a 24-well culture dish and incubated overnight at 4° C. with a final concentration of 10 μg/ml primary antibody to each well Ab diluent. Immunofluorescence with antibody Protein gene product 9.5 (PGP 9.5) (Biogenesis) was used to visualize neocortical neurite.

### Example 3

Microscopy and quantification of neurite growth. Culture cells were viewed on Nikon microscope lenses, and the images were recorded with a cooled CCD camera. On each coverslip, 30 cells were randomly selected. The total process length was measured by drawing all visible processes with IP Lab 3.6.5 software (Scanalytics, Inc). Processes shorter than cell diameter were excluded from the analysis. Data are expressed as the means±SEMs and reflect the results obtained from at least three independent experiments. Differences between groups were discriminated by one-way ANOVA. *p<0.05, **p<0.01 compared with the untreated control group.

### Example 4

Results. In the early development of neurons in culture, 3 h before plating, neurons start to extend dendrites to form lamellipodia (Moody and Heaton, Neuroscience 6:1707-1723, 1981; Bicknell and Beal, J Comp Neurol 226:508-522, 1984). They transform into distinct processes, which are similar in appearance and growth characteristics. 24 h after the minor processes appear, one of the minor processes begin to grow at much more rapid rate (Lesuisse and Martin, J Neurobiol 51:9-23, 2002; Dotti et al., J Neurosci 8:1454-1468, 1988). This process is axon, and it can be identified from dendrites by using different antibodies. In the control group, minor processes begin to grow at a rapid rate after 40 h. PbTx-2 in 30 nM concentration, however, begins to stimulate rapid growth of minor processes after 15 h (FIG. 1 C). Thus, PbTx-2 stimulates neurite growth in the early development of cortical neurons (FIG. 1 D), and also stimulates the rapid growth of one of minor processes for forming the axons (FIG. 1 C).

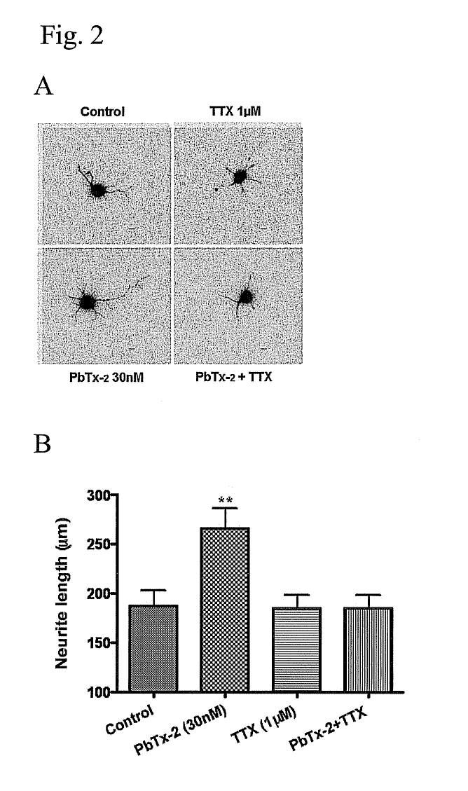

Neurons were treated in the absence and presence of tetradotoxin (TTX) in combination with 30 nM PbTx-2 (FIG. 2). PbTx-2 in the absence of TTX showed significant increases in the neurite growth (265.8±20.86 μm compared to 187.5±15.56 μm for control), whereas PbTx-2 in the presence of TTX showed inhibition of induced neurite growth to (190.2±10.76 μm). Therefore, PbTx-2 (30 nM) significantly increased NMDA activity, whereas no such effect was found in presence of TTX (FIG. 2).

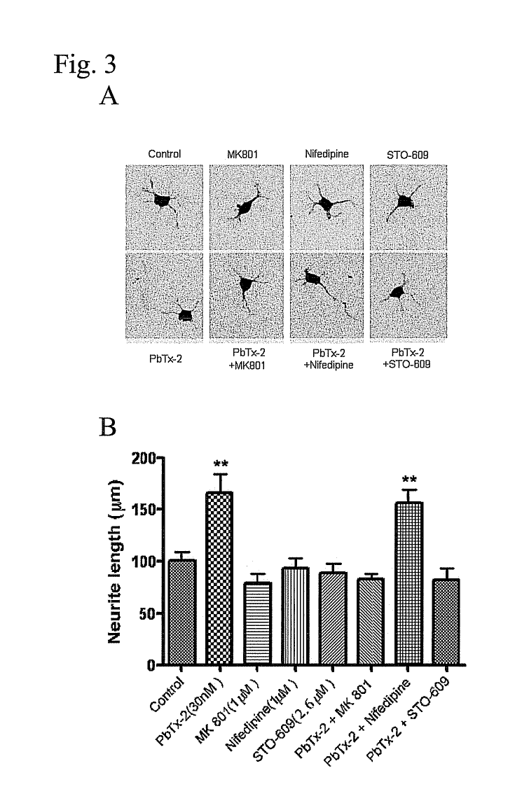

As shown in FIG. 3, brevetoxin-induced neurite growth was suppressed by MK801 from 166.5±17.89 μm (PbTx-2 alone) to 83.09±5.26 μm (PbTx-2 with MK801), control is 101.50±7.51 μm. Brevetoxin-induced neurite growth was not suppressed by nifedipine (156.60±12.48 μm). Further, STO-609 selectively blocked the stimulatory effect of brevetoxin from 166.50±17.89 μm (PbTx-2 alone) to 82.43±11.17 μm (PbTx-2 with STO-609). Note that PbTx-2-induced neurite growth, as is intracellular calcium increase, is blocked by MK801 and STO-609, indicating that it mediated by NMDAR and CaMKK pathway, not by VSCC.

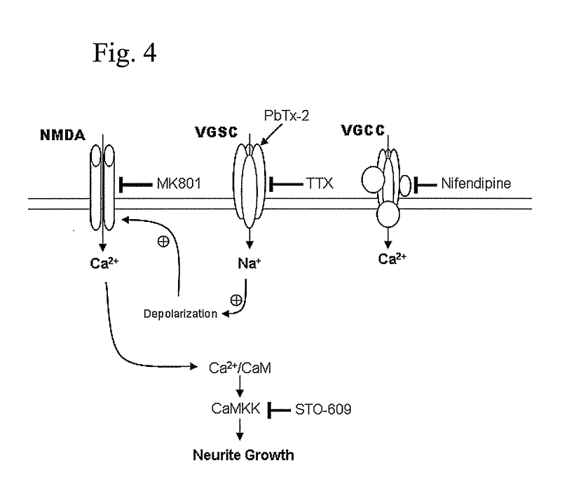

A signaling pathway triggered by brevotoxin that sequentially activates VGSC, NMDARs, CaMKK is illustrated in FIG. 4. Neurite growth is a complex response that depends on the Ca²⁺-dependent “cross-talk”. Brevetoxin acts to stimulate neurite growth by NMDA-mediated intracellular Ca²⁺ pathway, accelerating the neurons process development.

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A method of treatment of neurodegenerative diseases or disorders in a subject, comprising administering to a subject in need of such treatment a therapeutically effective amount of
a compound of formula I: 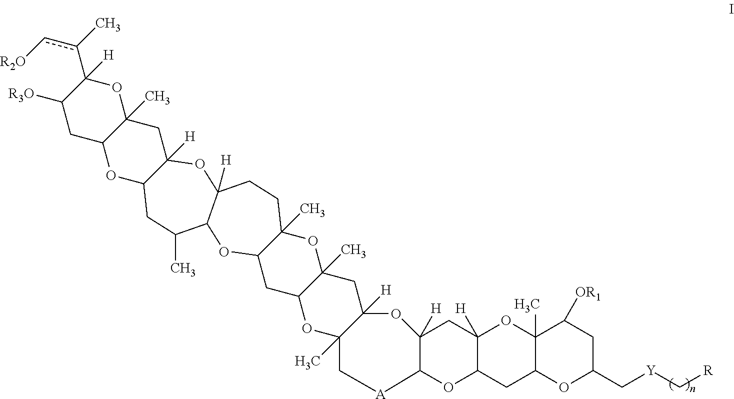
wherein
A is 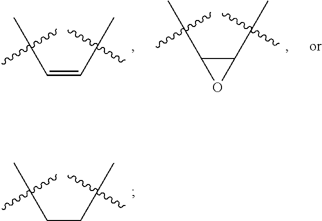
R is C₁-C₆ alkyl, C₂-C₆ alkenyl, C₁-C₆ alkyl esters, C₂-C₆ alkenyl esters, —CHO, —CO₂H, amines, amides, aryl esters, cycloalkyl esters, cycloalkenyl esters, purines, pyrimidines, heterocycle, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
R₁ is H or —(CO)CH₃; and
R₂ and R₃ at each occurrence are independently —CH₂(CO)CH₃, —CH₂(CO)CH₂CH₃, —CH₂(CO)CH(CH₃)₂, —CH₂(CO)CH₂CH₂CH₃, —CH₂(CO)CH(CH₃)CH₂CH₃, or —CH₂(CO)CH₂CH(CH₃)₂,
or OR₂ and OR₃ can be taken together to form a six membered ring of the formula (Ia) 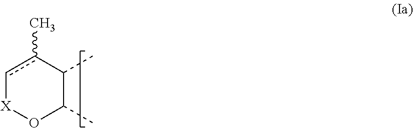
wherein X is C═O or CH(CH₃);
wherein the bracketed-dashed bonds indicate attachment to backbone; and
Y is CH═CH, C═CH₂, C═O, CHCH₃, or CH₂;
n is 0 or 1;
or a pharmaceutically acceptable salt, solvate, hydrate, complex, or combination thereof;
or a compound of formula III 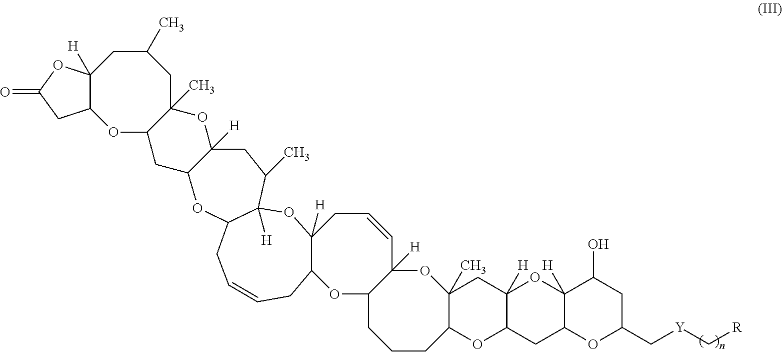
wherein
R is H, OH, halogen, C₁-C₆ lower alkyl, C₁-C₆ alkyl esters, C₂-C₆ alkenyl esters, amino, amido, formyl, carboxyl, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
Y is C═O, CH═CH, C═CH₂, CHCH₃ or CH₂; and
n is 0 or 1
or a pharmaceutically acceptable salt, solvate, hydrate, complex, or combination thereof.

2. (canceled)

3. A method of treatment of Alzheimer's Disease, Huntington's Disease, Parkinson's Disease, Multiple sclerosis (MS), Amyotrophic Lateral Sclerosis (ALS/Lou Gehrig's Disease) and other Motor Neuron Diseases, Prion Diseases, Frontotemporal Dementia (FTD), and CNS dysfunctions such as schizophrenia, depression, and epilepsy, neurodegenerations resulting from stroke, heart attack, head and spinal cord trauma, traumatic brain injury, bleeding in the brain and other injuries to the central nervous system (CNS) in a subject, comprising administering to a subject in need of such treatment a therapeutically effective amount of
a compound of formula I: 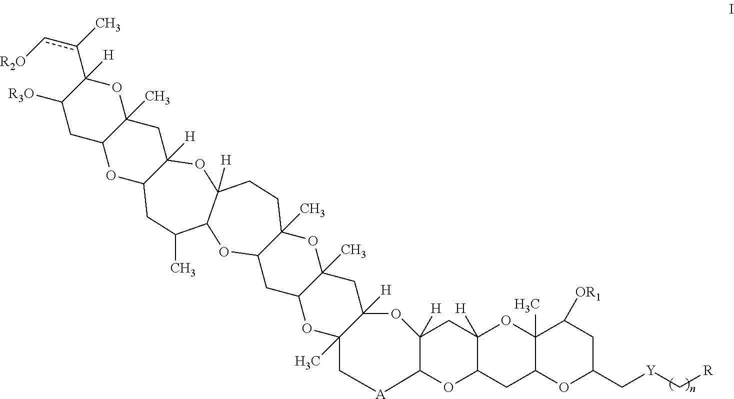
wherein
A is 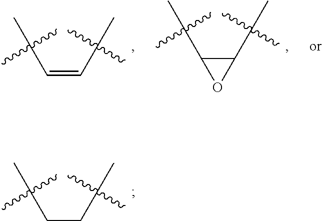
R is C₁-C₆ alkyl, C₂-C₆ alkenyl, C₁-C₆ alkyl esters, C₂-C₆ alkenyl esters, —CHO, —CO₂H, amines, amides, aryl esters, cycloalkyl esters, cycloalkenyl esters, purines, pyrimidines, heterocycle, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
R₁ is H or —(CO)CH₃; and
R₂ and R₃ at each occurrence are independently —CH₂(CO)CH₃, —CH₂(CO)CH₂CH₃, —CH₂(CO)CH(CH₃)₂, —CH₂(CO)CH₂CH₂CH₃, —CH₂(CO)CH(CH₃)CH₂CH₃, or —CH₂(CO)CH₂CH(CH₃)₂,
or OR₂ and OR₃ can be taken together to form a six membered ring of the formula (Ia) 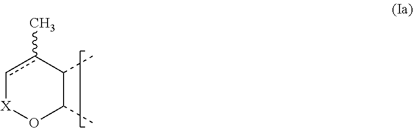
wherein X is C═O or CH(CH₃);
wherein the bracketed-dashed bonds indicate attachment to backbone; and
Y is CH═CH, C═CH₂, C═O, CHCH₃, or CH₂;
n is 0 or 1;
or a pharmaceutically acceptable salt, solvate, hydrate, complex, or combination thereof;
or a compound of formula III 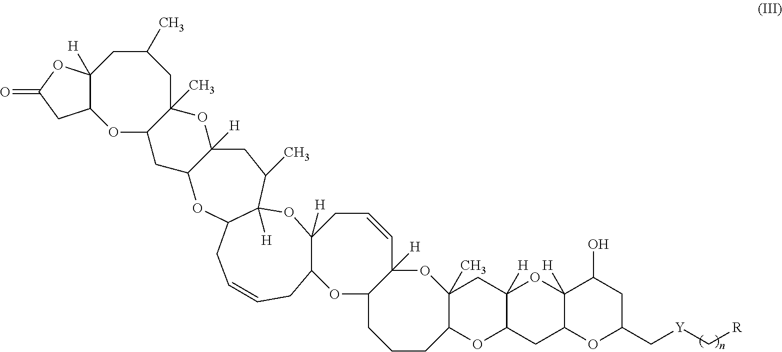
wherein
R is H, OH, halogen, C₁-C₆ lower alkyl, C₁-C₆ alkyl esters, C₂-C₆ alkenyl esters, amino, amido, formyl, carboxyl, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
Y is C═O, CH═CH, C═CH, CHCH₃ or CH₂; and
n is 0 or 1
or a pharmaceutically acceptable salt, solvate, hydrate, complex, or combination thereof.

4. (canceled)

5. A method of for enhancing neuronal growth comprising administering to a subject, or contacting a cell with, a compound in an amount effective to stimulate neuronal growth in the subject or cell, wherein
the compound is a compound of formula I: 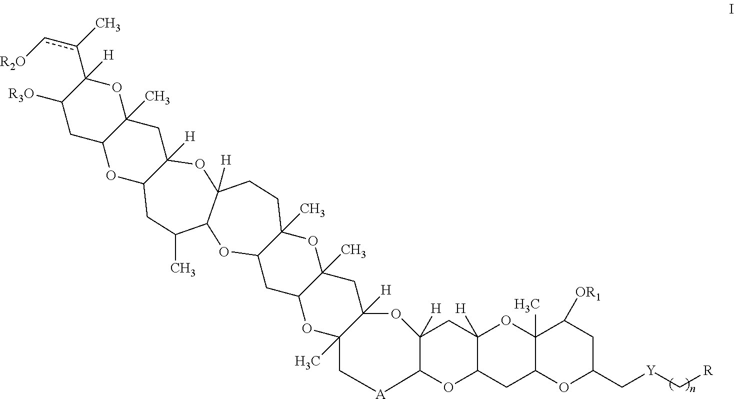
wherein
A is 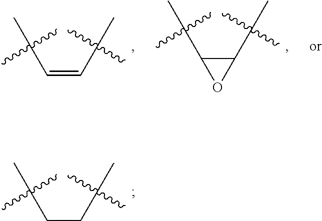
R is C₁-C₆ alkyl, C₂-C₆ alkenyl, C₁-C₆ alkyl esters, C₂-C₆ alkenyl esters, —CHO, —CO₂H, amines, amides, aryl esters, cycloalkyl esters, cycloalkenyl esters, purines, pyrimidines, heterocycle, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
R₁ is H or —(CO)CH₃; and
R₂ and R₃ at each occurrence are independently —CH₂(CO)CH₃, —CH₂(CO)CH₂CH₃, —CH₂(CO)CH(CH₃)₂, —CH₂(CO)CH₂CH₂CH₃, —CH₂(CO)CH(CH₃)CH₂CH₃, or —CH₂(CO)CH₂CH(CH₃)₂,
or OR₂ and OR₃ can be taken together to form a six membered ring of the formula (Ia) 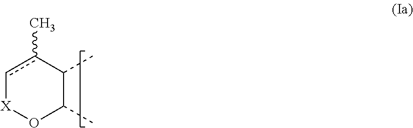
wherein X is C═O or CH(CH₃);
wherein the bracketed-dashed bonds indicate attachment to backbone; and
Y is CH═CH, C═CH₂, C═O, CHCH₃, or CH₂;
n is 0 or 1;
or a pharmaceutically acceptable salt, solvate, hydrate, complex, or combination thereof;
or the compound is a compound of formula III 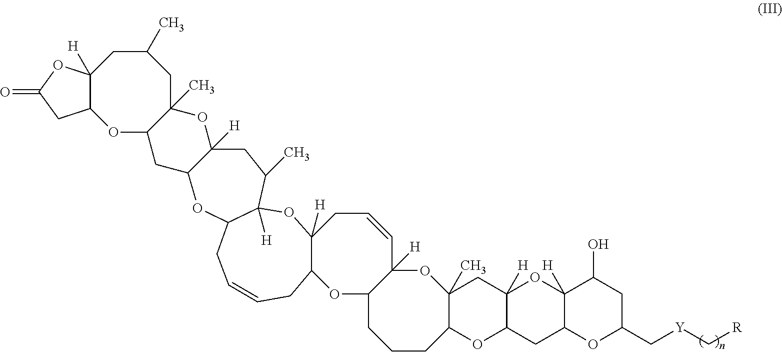
wherein
R is H, OH, halogen, C₁-C₆ lower alkyl, C₁-C₆ alkyl esters, C₂-C₆ alkenyl esters, amino, amido, formyl, carboxyl, aryl ester, cycloalkyl ester, cycloalkenyl ester, purinyl, pyrimidinyl, heterocyclyl, aryl, or heteroaryl, each of which is optionally substituted on any available carbon atom with C₁-C₁₀ alkyl, C₃-C₈ cycloalkyl, C₃-C₈ cycloalkyl(C₁-C₆)alkyl, C₃-C₈ cycloalkyl(C₁-C₆)alkoxy, C₁-C₁₀ alkoxy, halogen, hydroxy, cyano, nitro, amino, mono(C₁-C₆)alkylamino, di(C₁-C₆)alkylamino, C₂-C₁₀alkenyl, C₂-C₁₀alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl;
Y is C═O, CH═CH, C═CH₂, CHCH₃ or CH₂; and
n is 0 or 1
or a pharmaceutically acceptable salt, solvate, hydrate, complex, or combination thereof.

6. (canceled)

7. A method according to claim 1, wherein the compound is:

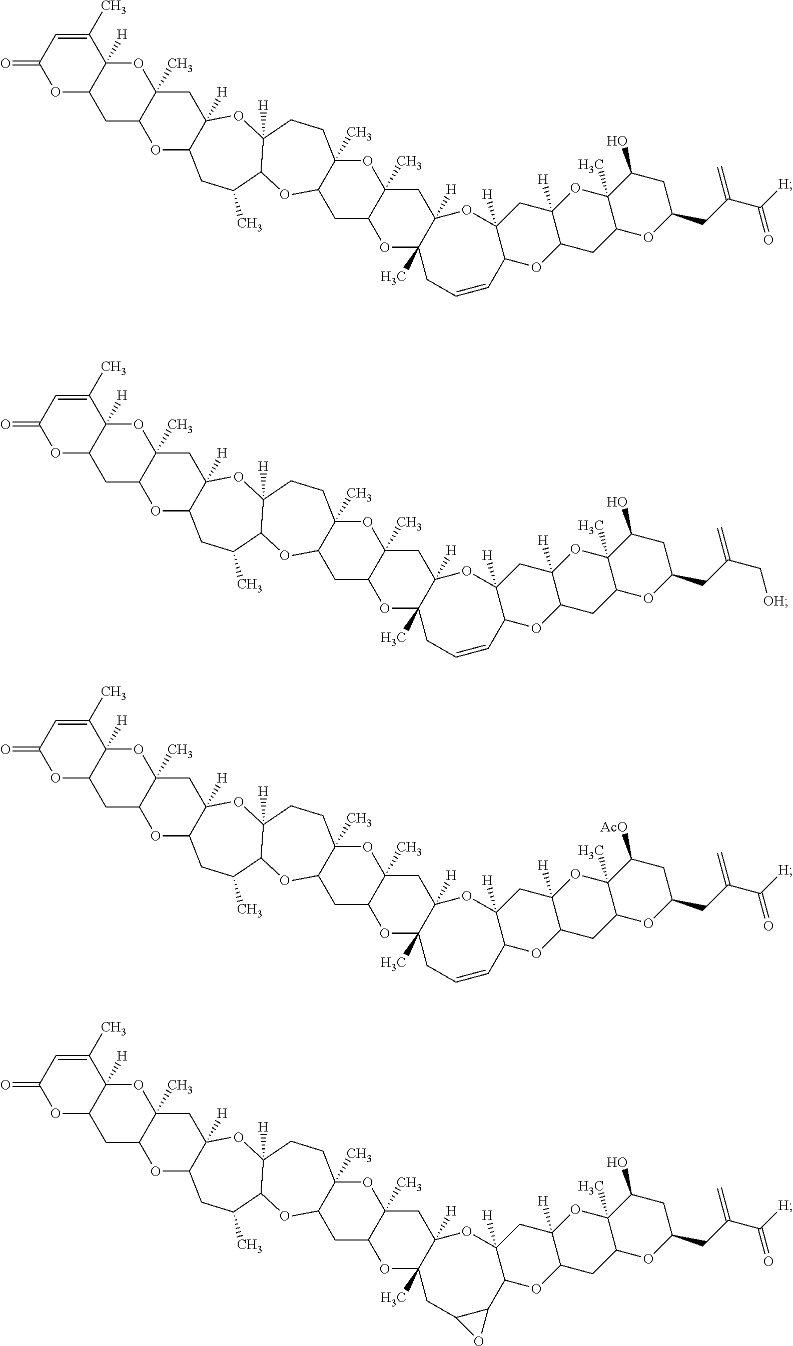

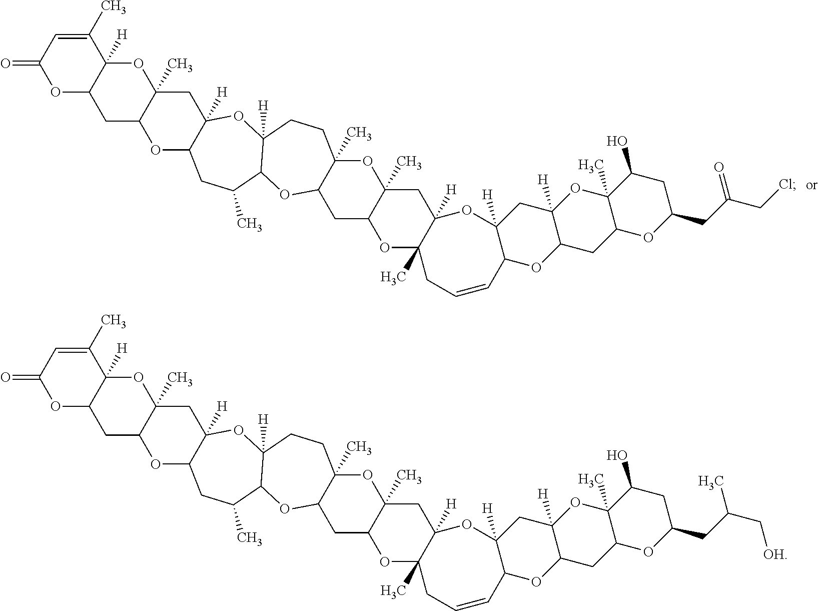

8. A method according to claim 1, wherein the compound is:

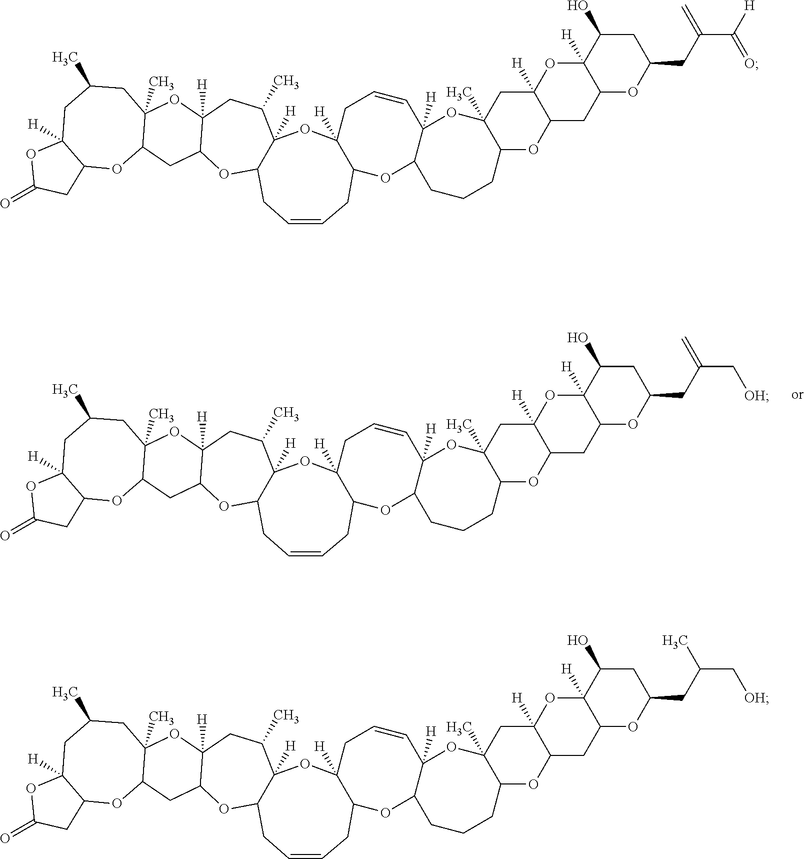

9. A method according to claim 3, wherein the compound is:

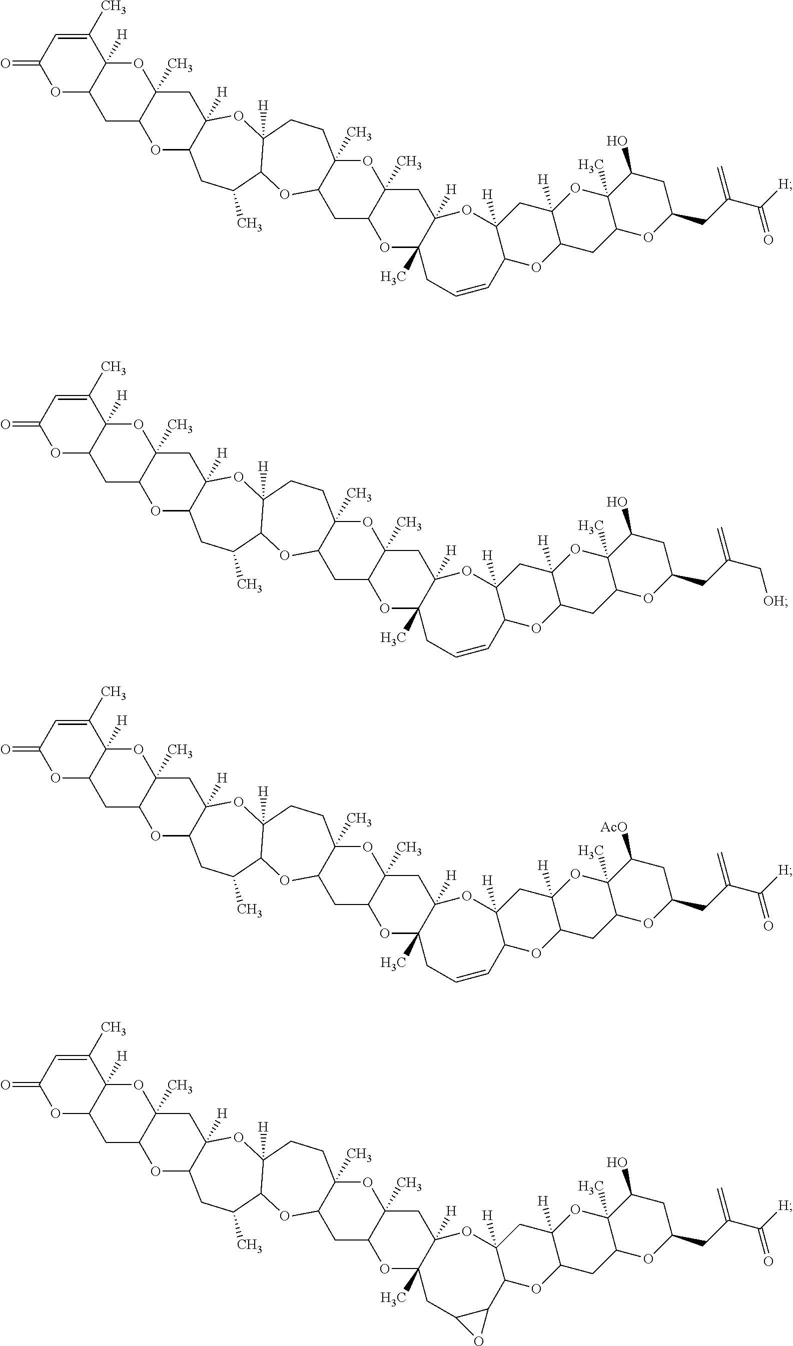

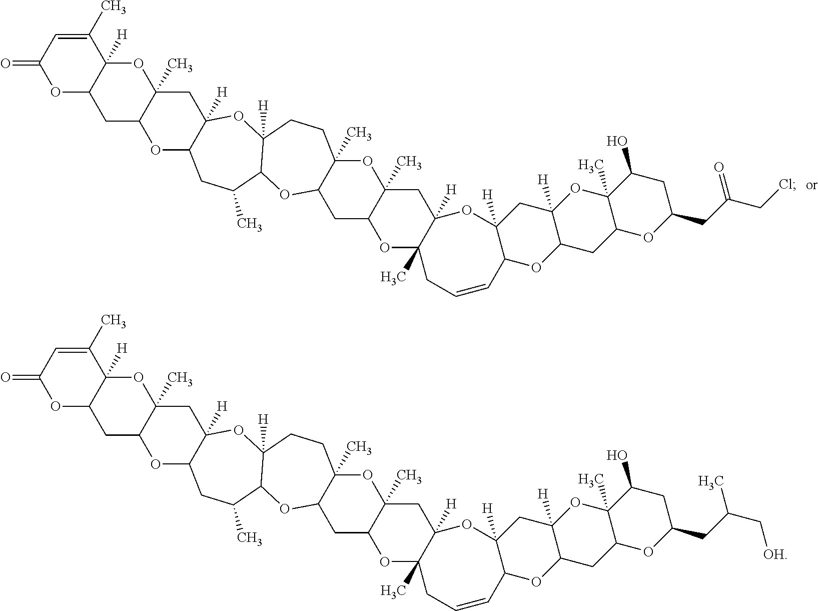

10. A method according to claim 3, wherein the compound is:

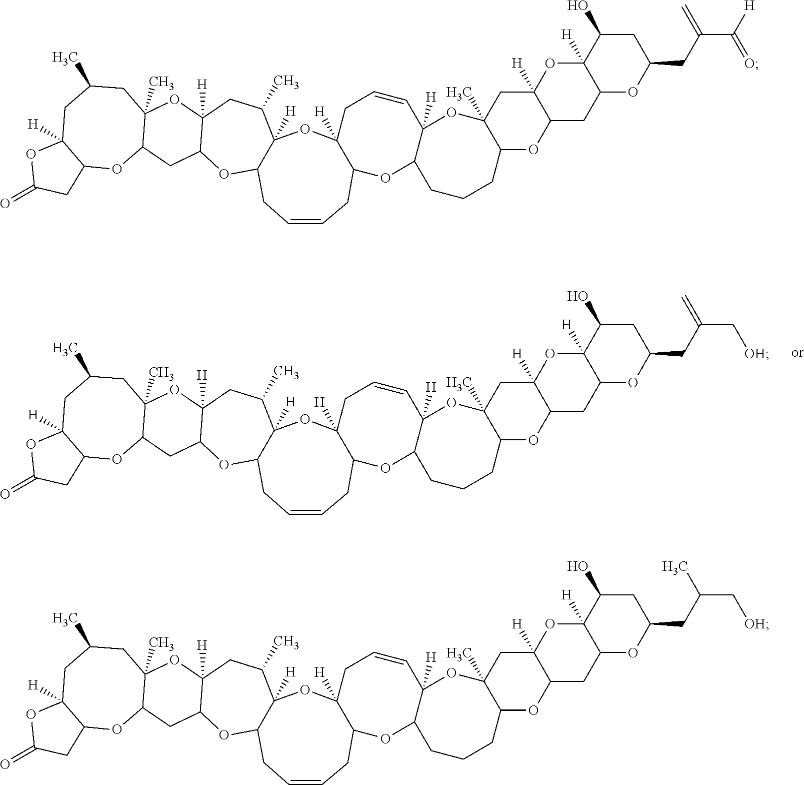

11. A method according to claim 5, wherein the compound is:

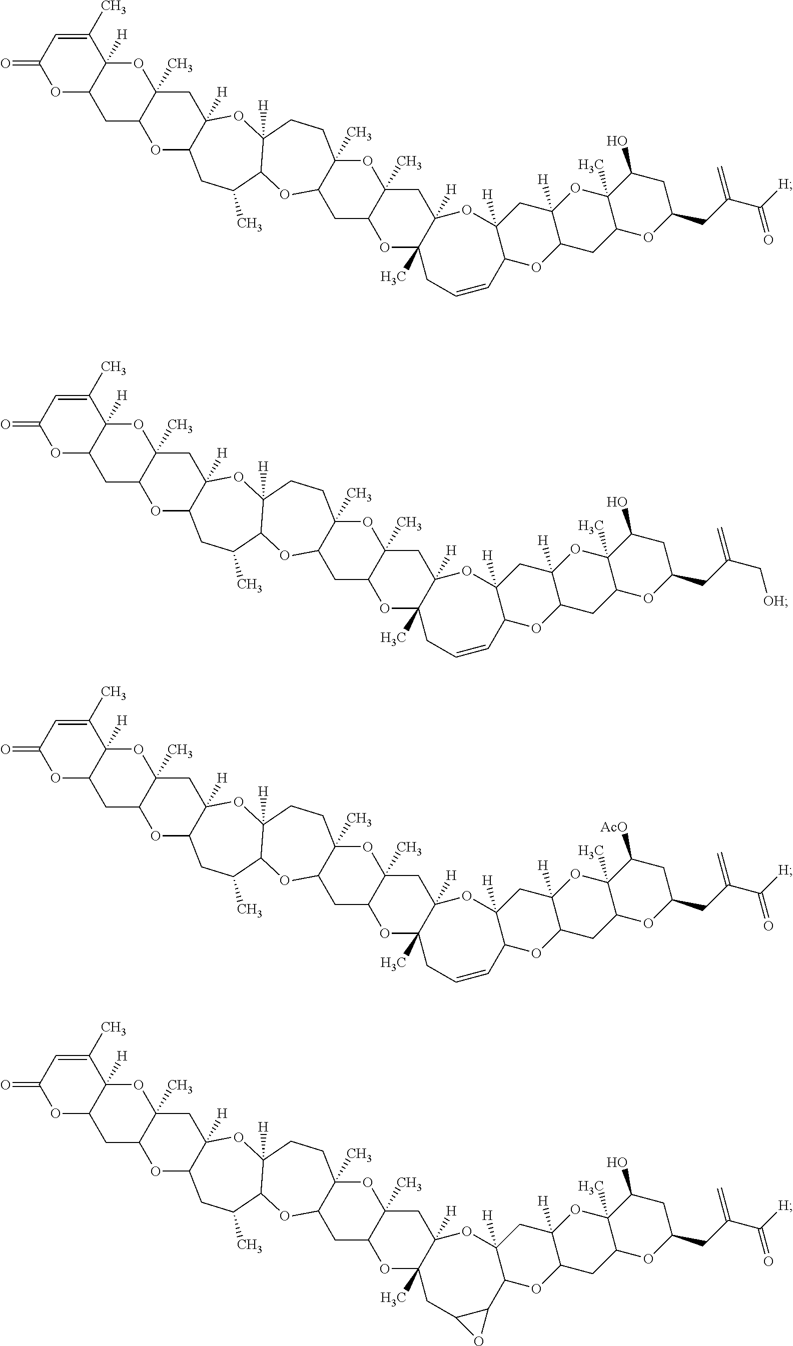

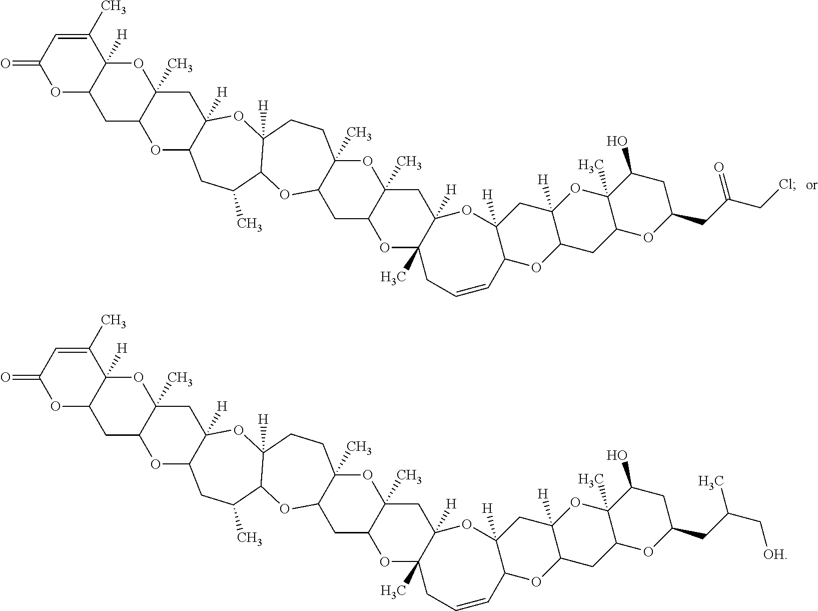

12. A method according to claim 5, wherein the compound is:

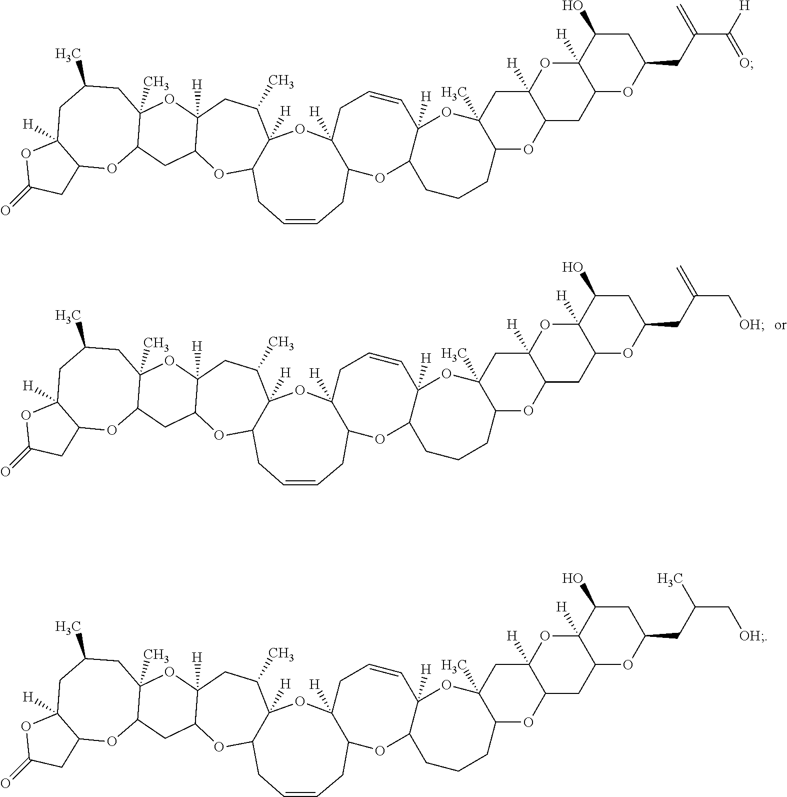

13. A method according to claim 1, wherein the therapeutically effective amount is between about 1 and 300 nM.

14. A method according to claim 1, wherein the therapeutically effective concentration is between about 10 and 100 nM

15. (canceled)

16. (canceled)

17. A method according to claim 1, wherein the therapeutically effective amount is non-lethal.

18. A method according to claim 1, wherein the therapeutically effective amount is nontoxic.

19. A method according to claim 3 wherein the therapeutically effective amount is between about 1 and 300 nM.

20. A method according to claim 5 herein the therapeutically effective amount is between about 1 and 300 nM.

21. A method according to claims 3 wherein the therapeutically effective amount is non-lethal.

22. A method according to claims 5 wherein the therapeutically effective amount is non-lethal.

23. A method according to claim 3 wherein the therapeutically effective amount is nontoxic.

24. A method according to claim 5, wherein the therapeutically effective amount is nontoxic.
